(19) United States
(12) Patent Application Publication
(10) Pub. No.: US 2023/0019141 A1
(43) Pub. Date: Jan. 19, 2023

(54) SYSTEM, METHOD, AND APPARATUS FOR PREDICTING GENETIC ANCESTRY

(71) Applicant: Mars, Incorporated, McLean, VA (US)

(72) Inventors: Daniel Garrigan, Vancouver, WA (US); Jason Huff, Vancouver, WA (US); Rebecca Chodroff Foran, Vancouver, WA (US)

(73) Assignee: Mars, Incorporated, McLean, VA (US)

(21) Appl. No.: 17/859,974

(22) Filed: Jul. 7, 2022

Related U.S. Application Data
(60) Provisional application No. 63/219,349, filed on Jul. 7, 2021.

(51) Int. Cl. G16B 10/00; G16B 40/20; G16B 20/20; G16B 20/40

(52) U.S. Cl. CPC G16B 10/00; G16B 40/20; G16B 20/20; G16B 20/40

(57) ABSTRACT

In one embodiment, a method includes accessing a sample of genetic material associated with a first animal, wherein the sample of genetic material comprises raw genotypes, generating phased haplotypes based on the raw genotypes, generating local assignments for genetic populations for the phased haplotypes by machine learning algorithms based on comparisons between the phased haplotypes and a reference panel comprising reference haplotypes associated with reference populations, and sending instructions to a user device for presenting an output associated with the first animal to a user, wherein the output is generated based on the local assignments for the genetic populations.

### CROSS-REFERENCE TO RELATED APPLICATIONS

This application claims the benefit of priority of U.S. Provisional Patent Application No. 63/219,349, filed Jul. 7, 2021, the content of which is incorporated herein by reference in its entirety, and to which priority is claimed.

### TECHNICAL FIELD

The embodiments described in the present disclosure relate to systems and methods for predicting the genetic ancestry of an animal based on input DNA sequences.

### BACKGROUND

Current methods of genetic mapping of animals suffer from an inability to assess admixed genome samples accurately and efficiently. Existing methods cannot efficiently process large numbers of query sequences, nor can they accurately provide the origin of certain provided samples. As a result, current analyses of the genomes of pets (and other domesticated animals) fail to achieve a satisfactory level of accuracy for both single-origin and admixed samples, resulting in wasted computational power and inaccurate results. The complexities associated with pet genomes are further complicated by the possibility of complex genetic profiles, which are the result of comingling of breeds. Given the ever-increasing complexity of downstream genetic profiles as well as the increased size and complexity of population genomic data sets, there is a need for systems and methods that can efficiently predict both local and global genetic ancestry of a given genome sample while retaining significant accuracy and without generating substantial computational costs.

Information regarding genetic risk factors for the development of diseases and of clinical and veterinarian recommendations can be helpful for the optimal management, monitoring and treatment of animals. Identification of ancestry contribution can be useful for the determination of these risk factors. Thus, there is a need for methods and systems to identify ancestry contributions accurately and efficiently.

### SUMMARY OF PARTICULAR EMBODIMENTS

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages, and in accordance with the purpose of the disclosed subject matter, as embodied and broadly described, the disclosed subject matter presents systems, methods, and apparatuses that can be used to collect, receive and/or analyze data. For example, certain non-limiting embodiments can be used to predict the genetic ancestry of an animal.

In certain non-limiting embodiments, the disclosure describes a system of computational and statistical methods for producing predictions of genetic ancestry and physical traits in companion animals from only their raw DNA sequences. The prediction system can utilize information from a large reference panel of animals with known genetic ancestry and traits to assign accurately genetic ancestry to small segments within the genome. The resulting segment classifications can be then aggregated on a per-animal basis and used to predict whether the individual animal belongs to one of hundreds of predefined purebred or admixed classes. In addition, the aggregate genetic ancestry classifications can be used to accurately predict physical traits, such as the adult body weight of the animal.

In certain non-limiting embodiments, one or more computing systems can access a sample of genetic material associated with a first animal. The sample of genetic material can comprise one or more raw genotypes. The computing systems can then generate one or more phased haplotypes based on the one or more raw genotypes. The computing systems can then generate, for the one or more phased haplotypes by one or more machine learning algorithms, one or more local assignments for one or more genetic populations based on comparisons between the one or more phased haplotypes and a reference panel comprising a plurality of reference haplotypes associated with a plurality of reference populations. The computing systems can further send, to a user device, instructions for presenting an output associated with the first animal to a user. In some embodiments, the output can be generated based on the one or more local assignments for the one or more genetic populations.

In certain non-limiting embodiments, one or more computer-readable non-transitory storage media embodying software is operable when executed to access a sample of genetic material associated with a first animal. The sample of genetic material can comprise one or more raw genotypes. The computer-readable non-transitory storage media embodying software is further operable when executed to generate one or more phased haplotypes based on the one or more raw genotypes. The computer-readable non-transitory storage media embodying software is further operable when executed to generate, for the one or more phased haplotypes by one or more machine learning algorithms, one or more local assignments for one or more genetic populations based on comparisons between the one or more phased haplotypes and a reference panel comprising a plurality of reference haplotypes associated with a plurality of reference populations. The computer-readable non-transitory storage media embodying software is further operable when executed to send, to a user device, instructions for presenting an output associated with the first animal to a user. In some embodiments, the output can be generated based on the one or more local assignments for the one or more genetic populations.

In certain non-limiting embodiments, a system can comprise one or more processors and a non-transitory memory coupled to the processors comprising instructions executable by the processors. The processors are operable when executing the instructions to access a sample of genetic material associated with a first animal. The sample of genetic material can comprise one or more raw genotypes. The processors are further operable when executing the instructions to generate one or more phased haplotypes based on the one or more raw genotypes. The processors are further operable when executing the instructions to generate, for the one or more phased haplotypes by one or more machine learning algorithms, one or more local assignments for one or more genetic populations based on comparisons between the one or more phased haplotypes and a reference panel comprising a plurality of reference haplotypes associated with a plurality of reference populations. The processors are further operable when executing the instructions to send, to a user device, instructions for presenting an output associated with the first animal to a user. In some embodiments, the output can be generated based on the one or more local assignments for the one or more genetic populations.

Furthermore, the disclosed embodiments of the methods, computer readable non-transitory storage media, and systems can have further non-limiting features as described below.

In certain non-limiting embodiments, the computing systems can further generate, based on the one or more raw genotypes, one or more consensus genotypes. The computing systems can then generate, based on the one or more raw genotypes and the one or more consensus genotypes, the one or more phased haplotypes. In some embodiments, the generating can comprise phasing the one or more raw genotypes and the one or more consensus genotypes into maternal and paternal chromosomes. In one feature, the one or more machine learning algorithms can comprise a positional Burrows-Wheeler transform algorithm.

In certain non-limiting embodiments, the computing systems can remove one or more errors associated with the one or more local assignments for the one or more genetic populations based on the one or more machine learning algorithms. In one feature, the one or more machine learning algorithms can comprise a hidden Markov model.

In certain non-limiting embodiments, the computing systems can further determine, based on the one or more local assignments for the one or more genetic populations, one or more source populations associated with the first animal. In some embodiments, determining the one or more source populations can comprise aggregating the one or more local assignments for the one or more genetic populations over both maternal and paternal chromosomes, calculating proportions associated with the one or more source populations based on the aggregations, and determining the one or more source populations based on the calculated proportions.

In certain non-limiting embodiments, the computing systems can further partition the one or more local assignments for the one or more genetic populations into one or more of a maternally-inherited group or a paternally-inherited group. The partitioning can be based on one or more clustering algorithms.

In certain non-limiting embodiments, the computing systems can further determine, based on the one or more local assignments for the one or more genetic populations and the one or more source populations, one or more genetic traits associated with the first animal. In some embodiments, determining the one or more genetic traits can be further based on one or more of genotypes of variants of large effect, genome-wide statistics, genomic principal component analysis (PCA) projections, DNA methylation profiles, or polygenic risk scores. In some embodiments, the one or more genetic traits comprise one or more of a range of adult body weight, a risk prediction or a predisposition to a genetic disease, a nutrition recommendation, a behavior and temperament class prediction, a longevity estimation, an all-causes mortality prediction in years, a predicted pharmacological response, or a recovery time range in hours for injectable anesthetics.

In certain non-limiting embodiments, the computing systems can further update the one or more machine learning algorithms based one or more new reference samples added to the reference panel. In some embodiments, the updating can comprise applying a cross-validation across all samples in the reference panel, identifying, based on results associated with the cross-validation by a detection algorithm, one or more outliers, and removing the identified outliers from the reference panel. In some embodiments, the updating can further comprise generating one or more labels for one or more unlabeled samples in the reference panel, wherein the updating is based on the generated labels. The updating can be repeatedly iterated until a predetermined accuracy level of the one or more machine learning algorithms is reached.

In certain non-limiting embodiments, the present disclosure provides a kit for determining local ancestry and global ancestry of an animal with any of the method disclosed herein. In certain embodiments, the kit comprises a sample collection device. In certain embodiments, the sample collection device comprises a carrier and a reservoir. In certain embodiments, the carrier comprises an absorbent member and wherein the reservoir comprises a shield. In certain embodiments, the kit further comprises written instructions on how to use the sample collection device and/or how to collect a sample.

In certain non-limiting embodiments, one or more computing systems can access a sample of genetic material associated with a first animal. The sample of genetic material can comprise one or more raw genotypes. The computing systems can then generate one or more phased haplotypes based on the one or more raw genotypes. The computing systems can then generate, for the one or more phased haplotypes by one or more machine learning algorithms, one or more local assignments for one or more genetic populations based on comparisons between the one or more phased haplotypes and a reference panel comprising a plurality of reference haplotypes associated with a plurality of reference populations. The computing systems can then determine, based on the one or more local assignments for the one or more genetic populations, one or more source populations associated with the first animal. The computing systems can then partition the one or more local assignments for the one or more genetic populations into one or more of a maternally-inherited group or a paternally-inherited group. The computing systems can then determine, based on the one or more local assignments for the one or more genetic populations and the one or more source populations, one or more genetic traits associated with the first animal. The computing systems can further send, to a user device, instructions for presenting an output associated with the first animal to a user. In some embodiments, the output can be generated based on one or more of the one or more local assignments for the one or more genetic populations, the one or more source populations, results associated with the partitioning, or the one or more genetic traits.

In certain non-limiting embodiments, one or more computer-readable non-transitory storage media embodying software is operable when executed to access a sample of genetic material associated with a first animal. The sample of genetic material can comprise one or more raw genotypes. The computer-readable non-transitory storage media embodying software is further operable when executed to generate one or more phased haplotypes based on the one or more raw genotypes. The computer-readable non-transitory storage media embodying software is further operable when executed to generate, for the one or more phased haplotypes by one or more machine learning algorithms, one or more local assignments for one or more genetic populations based on comparisons between the one or more phased haplotypes and a reference panel comprising a plurality of reference haplotypes associated with a plurality of reference populations. The computer-readable non-transitory storage media embodying software is further operable when executed to determine, based on the one or more local assignments for the one or more genetic populations, one or more source populations associated with the first animal. The computer-readable non-transitory storage media embodying software is further operable when executed to partition the one or more local assignments for the one or more genetic populations into one or more of a maternally-inherited group or a paternally-inherited group. The computer-readable non-transitory storage media embodying software is further operable when executed to determine, based on the one or more local assignments for the one or more genetic populations and the one or more source populations, one or more genetic traits associated with the first animal. The computer-readable non-transitory storage media embodying software is further operable when executed to send, to a user device, instructions for presenting an output associated with the first animal to a user. In some embodiments, the output can be generated based on one or more of the one or more local assignments for the one or more genetic populations, the one or more source populations, results associated with the partitioning, or the one or more genetic traits.

In certain non-limiting embodiments, a system can comprise one or more processors and a non-transitory memory coupled to the processors comprising instructions executable by the processors. The processors are operable when executing the instructions to access a sample of genetic material associated with a first animal. The sample of genetic material can comprise one or more raw genotypes. The processors are further operable when executing the instructions to generate one or more phased haplotypes based on the one or more raw genotypes. The processors are further operable when executing the instructions to generate, for the one or more phased haplotypes by one or more machine learning algorithms, one or more local assignments for one or more genetic populations based on comparisons between the one or more phased haplotypes and a reference panel comprising a plurality of reference haplotypes associated with a plurality of reference populations. The processors are further operable when executing the instructions to determine, based on the one or more local assignments for the one or more genetic populations, one or more source populations associated with the first animal. The processors are further operable when executing the instructions to partition the one or more local assignments for the one or more genetic populations into one or more of a maternally-inherited group or a paternally-inherited group. The processors are further operable when executing the instructions to determine, based on the one or more local assignments for the one or more genetic populations and the one or more source populations, one or more genetic traits associated with the first animal. The processors are further operable when executing the instructions to send, to a user device, instructions for presenting an output associated with the first animal to a user. In some embodiments, the output can be generated based on one or more of the one or more local assignments for the one or more genetic populations, the one or more source populations, results associated with the partitioning, or the one or more genetic traits.

Furthermore, the disclosed embodiments of the methods, computer readable non-transitory storage media, and systems can have further non-limiting features as described below.

In certain non-limiting embodiments, the computing systems can further generate, based on the one or more raw genotypes, one or more consensus genotypes. The computing systems can then generate, based on the one or more raw genotypes and the one or more consensus genotypes, the one or more phased haplotypes. In some embodiments, the generating can comprise phasing the one or more raw genotypes and the one or more consensus genotypes into maternal and paternal chromosomes. In one feature, the one or more machine learning algorithms can comprise a positional Burrows-Wheeler transform algorithm.

In certain non-limiting embodiments, the computing systems can remove one or more errors associated with the one or more local assignments for the one or more genetic populations based on the one or more machine learning algorithms. In one feature, the one or more machine learning algorithms can comprise a hidden Markov model.

In certain non-limiting embodiments, determining the one or more source populations can comprise aggregating the one or more local assignments for the one or more genetic populations over both maternal and paternal chromosomes, calculating proportions associated with the one or more source populations based on the aggregations, and determining the one or more source populations based on the calculated proportions. In some embodiments, the partitioning can be based on one or more clustering algorithms.

In certain non-limiting embodiments, determining the one or more genetic traits can be further based on one or more of genotypes of variants of large effect, genome-wide statistics, genomic principal component analysis (PCA) projections, DNA methylation profiles, or polygenic risk scores. In some embodiments, the one or more genetic traits comprise one or more of a range of adult body weight, a risk prediction or a predisposition to a genetic disease, a nutrition recommendation, a behavior and temperament class prediction, a longevity estimation, an all-causes mortality prediction in years, a predicted pharmacological response, or a recovery time range in hours for injectable anesthetics.

In certain non-limiting embodiments, the computing systems can further update the one or more machine learning algorithms based one or more new reference samples added to the reference panel. In some embodiments, the updating can comprise applying a cross-validation across all samples in the reference panel, identifying, based on results associated with the cross-validation by a detection algorithm, one or more outliers, and removing the identified outliers from the reference panel. In some embodiments, the updating can further comprise generating one or more labels for one or more unlabeled samples in the reference panel, wherein the updating is based on the generated labels. The updating can be repeatedly iterated until a predetermined accuracy level of the one or more machine learning algorithms is reached.

It is to be understood that both the foregoing general description and the following detailed description are exemplary and are intended to provide further explanation of the disclosed subject matter claimed. These and other features, aspects, and advantages of the disclosure will be apparent from a reading of the following detailed description together with the accompanying drawings, which are briefly described below. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise.

### BRIEF DESCRIPTION OF THE DRAWINGS

The foregoing and other objects, features, and advantages of the disclosure will be apparent from the following description of embodiments as illustrated in the accompanying drawings, in which reference characters refer to the same parts throughout the various views. The drawings are not necessarily to scale, emphasis instead being placed upon illustrating principles of the disclosure:

FIG. 1 illustrates an exemplary workflow for the system according to the presently disclosed subject matter;

FIG. 2 illustrates an example workflow of the local ancestry classifier;

FIG. 3 illustrates a plurality of models showing the results of varying the predetermined subregion length, between 6 centimorgans and 48 centimorgans;

FIG. 4 illustrates an example marginal match length according to the presently disclosed subject matter;

FIG. 5 illustrates an example comparison between a “chromosome painting” model (A) and the PBWT-based model described in this disclosure (B and C);

FIG. 6 illustrates an example smoothing process;

FIG. 7A illustrates a confusion matrix related to a plurality of animal species and/or of animal breeds; FIG. 7B shows animal breeds of the y-axis; FIG. 7C shows animal breed of the x-axis;

FIG. 8 illustrates an example sort of chromosome pairs into maternal and paternal copies using k-means clustering;

FIG. 9 illustrates example principal components from global ancestry proportions for a set of chromosomes;

FIG. 10 illustrates example results of accuracy benchmark of the presently disclosed system versus the state-of-the-art classifier RFMix;

FIG. 11 illustrates an example receiver operating characteristic (ROC) curve for the global ancestry classifier;

FIG. 12 illustrates an example regression of predicted adult body weight and true observed adult body weight;

FIG. 13 illustrates an example iterative improvement of local ancestry reference panel using the isolation forest technique for anomaly detection; and

FIG. 14 illustrates an example method for ancestry prediction.

### DESCRIPTION OF EXAMPLE EMBODIMENTS

The mapping of local and global genetic ancestry traits within a population of pets is an ongoing aspect of population genetics research. In this context, the term “ancestry” refers to the source population from which a segment of DNA is derived. Furthermore, the qualification “local ancestry” refers to the source population for a small segment of DNA that makes up a chromosome. Alternatively, the qualification “global ancestry” refers to one or more source populations that contribute to the totality of all chromosomes. While local ancestry can assign a single source population to a localized segment of DNA, global ancestry can describe the aggregation of local ancestries across all DNA segments in the genome. Global ancestry can be reported as proportions of an organism's genome derived from given source populations. Importantly, both local and global ancestry classification can depend on a reference panel that typifies the DNA segments of all source populations. As the sample size of population genomic data increases, it can become more computationally complex to assign new sequences to predefined population groups. In particular, with respect to many pets, such as cats, dogs, and other domesticated animals, genome sequences can become admixed, as subsequent generations interbreed and create more complicated genomes.

There remains a need in the art for systems and methods that can accurately and efficiently predict the genetic ancestry of a query sample, whether the sample is of a single-origin or admixed, and which can be scaled. The presently disclosed subject matter addresses this need through the following methods and systems.

Certain systems and methods according to the present embodiments use computational and statistical methods for producing predictions of genetic ancestry and physical traits in companion animals from only their raw DNA sequences. The systems and methods can ingest batches of DNA sequences from a set of samples with unknown genetic ancestry and then efficiently matching this “query” set to a curated reference database of DNA sequences with known genetic ancestry and traits. In particular embodiments, information from a large reference panel of animals with known genetic ancestry and traits can be used to assign accurately genetic ancestry to small segments within the genome. The resulting segment classifications can be then aggregated on a per-animal basis and used to predict whether the individual animal belongs to one of hundreds of predefined purebred or admixed classes. In addition, the aggregate genetic ancestry classifications can be also used to accurately predict physical traits, such as the adult body weight of the animal. The details of the present embodiments are provided below. For clarity and not by way of limitation, the detailed description of the present disclosure is divided into the following subsections:
1. Definitions;
2. Overview of the System;
3. Sequencing, Kits, and Methods of Treatment; and
4. Examples.

### 1. DEFINITIONS

The terms used in this specification generally have their ordinary meanings in the art, within the context of this disclosure and in the specific context where each term is used. Certain terms are discussed below, or elsewhere in the specification, to provide additional guidance in describing the compositions and methods of the disclosure and how to make and use them.

Unless defined otherwise, all technical and scientific terms used herein have the meaning commonly understood by a person skilled in the art to which this invention belongs. The following references provide one of skill with a general definition of many of the terms used in the present disclosure: King, Mulligan, and Stansfield. A Dictionary of Genetics, Oxford University Press, 2013; Glossary of Bioinformatics Terms, Current Protocols in Bioinformatics, 35, 1934-3396, 2011; and Whole-Transcriptome Amplification of Single Cells for Next-Generation Sequencing, Current Protocols in Molecular Biology, 111, 1934-3639, 2015. As used herein, the following terms have the meanings ascribed to them below, unless specified otherwise.

As used herein, the words “a” or “an,” when used in conjunction with the term “comprising” in the claims and/or the specification, can mean “one,” but they are also consistent with the meaning of “one or more,” “at least one,” and/or “one or more than one.” Furthermore, the terms “having,” “including,” “containing” and “comprising” are interchangeable, and one of skill in the art will recognize that these terms are open ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to systems or processes, the term can mean within an order of magnitude, preferably within 5-fold, and more preferably within 2-fold, of a value.

As used herein, the terms “comprises,” “comprising,” or any other variation thereof, are intended to cover a non-exclusive inclusion, such that a process, method, article, or apparatus that comprises a list of elements does not include only those elements but can include other elements not expressly listed or inherent to such process, method, article, or apparatus.

As used herein, the term “local ancestry” refers to ancestral origin of distinct chromosomal segments within an individual genome. In certain embodiments, local ancestry is the call for a specific segment of a chromosome in an animal, e.g., breed of a dog. In certain exemplary embodiments, local ancestry refers to the genetic ancestry of an individual at a particular chromosomal location, where an individual can have 0, 1 or 2 copies of an allele derived from each ancestral population.

As used herein, the term “global ancestry” refers to ancestry proportions averaged across the genome of a subject. In certain embodiments, global ancestry is the proportion of calls over the entire genome of an animal, e.g., breeds of a dog.

As used herein, the term “haplotype” refers to a set of linked genes or other genetic markers that are inherited together as a unit. During meiosis there is little or no recombination with the corresponding region on the homologous chromosome, and hence shuffling of alleles between the homologous regions is rare. In certain embodiments, the stretch of DNA containing a haplotype is called a “haplotype block”. For example, without any limitation, certain genes of the major histocompatibility complex in canines are closely linked at the DLA locus on chromosome 12 and behave as a haplotype, with the alleles on maternal and paternal chromosomes generally transmitted to offspring in the same combinations. In certain embodiments, the term “haplotype” refers to a single chromosome or to a haploid set of chromosomes. As used herein, the term “haplotype estimation” or “haplotype phasing” refers to the process of statistical estimation of haplotypes from genotype data.

As used herein, the terms “centimorgan” or “cM” refer to a unit of measure for the frequency of genetic recombination. One centimorgan is equal to a 1% chance that two markers on a chromosome will become separated from one another due to a recombination event during meiosis (which occurs during the formation of egg and sperm cells). On average, one centimorgan corresponds to roughly 1 million base pairs in the human genome.

As used herein, the term “phasing” refers to the process of assigning alleles (e.g., A, C, T, and G) to the paternal and maternal chromosomes. The term is usually applied to types of DNA that recombine (e.g., autosomal DNA or the X chromosome). In certain embodiments, phasing can help to determine whether matches are on the paternal side or the maternal side, on both sides or on neither side. In certain embodiments, phasing can also help with the process of chromosome mapping (e.g., assigning segments to specific ancestors). Conventionally, the use of phased data reduces the number of false positive matches.

As used herein, the term “genotype” refers to the genetic makeup of an organism. For example, the genotype describes the complete set of genes of an organism, e.g., dog. In certain embodiments, the term “genotype” refers to the alleles, or variant forms of a gene, that are carried by an organism. A particular genotype is described as homozygous if it features two identical alleles and as heterozygous if the two alleles differ. As used herein, the process of determining a genotype is called “genotyping.” As used herein, the term “genotype calling” and variations thereof refers to estimating genotype values from raw or processed data.

The terms “nucleic acid molecule,” “nucleotide sequence” and “polynucleotide,” as used herein, refer to a single or double stranded covalently linked sequence of nucleotides in which the 3′ and 5′ ends on each nucleotide are joined by phosphodiester bonds. The nucleic acid molecule can include deoxyribonucleotide bases or ribonucleotide bases and can be manufactured synthetically in vitro or isolated from natural sources.

The terms “polypeptide,” “peptide,” “amino acid sequence” and “protein,” used interchangeably herein, refer to a molecule formed from the linking of at least two amino acids. The link between one amino acid residue and the next is an amide bond and is sometimes referred to as a peptide bond. A polypeptide can be obtained by a suitable method known in the art, including isolation from natural sources, expression in a recombinant expression system, chemical synthesis or enzymatic synthesis. The terms can apply to amino acid polymers in which one or more amino acid residue is an artificial chemical mimetic of a corresponding naturally occurring amino acid, as well as to naturally occurring amino acid polymers and non-naturally occurring amino acid polymers.

The term “pet food” or “pet food composition” or “pet food product” or “final pet food product” means a product or composition that is intended for consumption by, and provides certain nutritional benefit to a companion animal, such as a cat, a dog, a guinea pig, a rabbit, a bird or a horse. For example, but not by way of limitation, the companion animal can be a “domestic” dog, e.g., Canis lupus familiaris. In certain embodiments, the companion animal can be a “domestic” cat such as Felis domesticus. A “pet food” or “pet food composition” or “pet food product” or “final pet food product” includes any food, feed, snack, food supplement, liquid, beverage, treat, toy (chewable and/or consumable toys), meal substitute or meal replacement.

For the purposes of this disclosure the term “user”, “subscriber” “consumer” or “customer” should be understood to refer to a user of an application or applications as described herein and/or a consumer of data supplied by a data provider. By way of example, and not limitation, the term “user” or “subscriber” can refer to a person who receives data provided by the data or service provider over the Internet in a browser session, or can refer to an automated software application which receives the data and stores or processes the data.

### 2. OVERVIEW OF SYSTEM

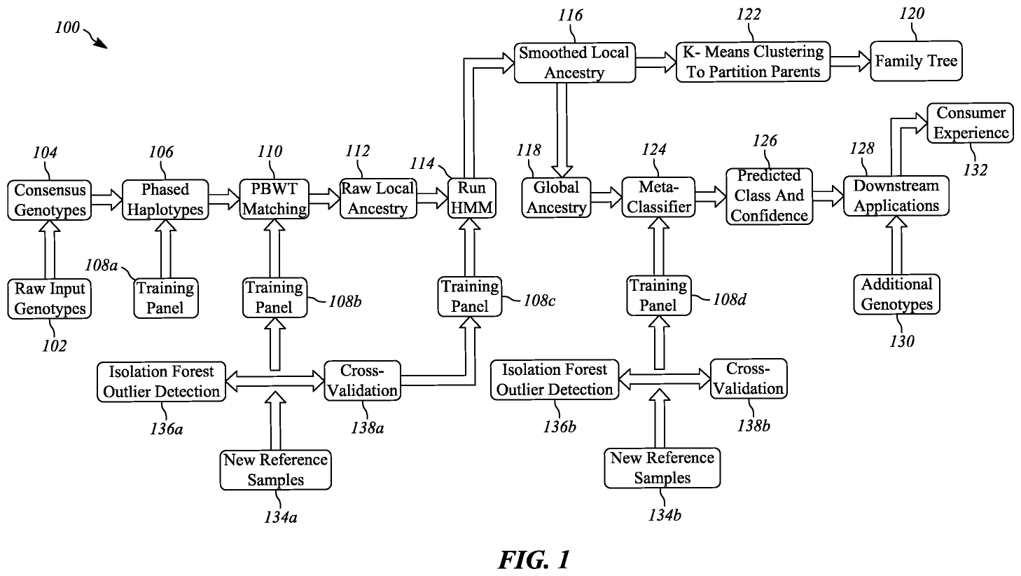

FIG. 1 illustrates an exemplary workflow 100 for the system according to the presently disclosed subject matter. The system can ingest batches of DNA sequences from a set of samples with unknown genetic ancestry and then efficiently match this “query” set to a curated reference database of DNA sequences with known genetic ancestry and traits. In certain embodiments, the DNA sequences encompassed by the present disclosure include gene sequences and/or genetic markers. For example, but without any limitation, genetic markers include single nucleotide polymorphisms (SNPs), short tandem repeats (STRs), insertions and deletions of bases (indels), and copy number variants (CNVs).

In particular embodiments, the system can comprise a plurality of individual component subsystems. These subsystems can comprise one or more of a local ancestry classifier, a global ancestry classifier, a predictor of genealogical ancestry, a predictor of traits suite (e.g., physical, behavioral, and metabolic), or an automated system for accuracy improvement of the above classifiers. These subsystems can have their own respective functionalities. When combined, these subsystems can enable the entire system to produce predictions of genetic ancestry and physical traits in companion animals from only their raw DNA sequences.

In certain non-limiting embodiments, the local ancestry classifier can be associated with the raw input genotypes 102, the consensus genotypes 104, the phased haplotypes 106, the train panel 108a-108c, the PBWT matching 110, the raw local ancestry 112, the HMM 114, and the smoothed local ancestry 116. The local ancestry classifier can take a raw input genotype 102 and generate a consensus genotype 104 accordingly. In some embodiments, the raw input genotype 102 can act as a query genotype and the consensus genotype 104 can act as a reference genotype. The consensus genotype 104 can be then processed into a phased haplotype 106, which can distinguish between maternal and paternal chromosomes. A matching process 110 (e.g., a positional Burrows-Wheeler-transform) can then partition the phased haplotype 106 into a plurality of windows, which can be compared against a reference or training panel 108. The density of matches between the phased haplotype 106 and the reference or training panel 108 can be calculated, and can produce a raw local ancestry 112, which can be defined as the reference population having the highest relative density of matches (or other criteria). The raw local ancestry 112 can then be used as an input to a hidden Markov model (HMM) 114, which can remove or replace certain errors within the raw local ancestry 112 in order to produce a smoothed local ancestry 116. This smoothed local ancestry 116 can be output to an end user, to show the relative origin of one or more chromosomes. As an example and not by way of limitation, the output can comprise detailed descriptions of an animal's chromosomes, showing exactly where the animal got each piece of the DNA, e.g., Great Pyrenees, German Shepherd Dog, Beauceron, White Swiss Shepherd, Maremma Sheepdog, Chow Chow, Siberian Husky, Parson Russell Terrier, Border Terrier, and Hovawart.

In certain non-limiting embodiments, the global ancestry classifier can then use the smoothed local ancestry 116 to generate a global ancestry 118. This global ancestry 118 can be output to an end user, providing the relative contribution of different origin populations in the animal's genome. As an example and not by way of limitation, the output can comprise different breeds detected in the animal's DNA.

In certain non-limiting embodiments, the predictor of genealogical ancestry can use the smoothed local ancestry 116 to predict genealogical ancestry. As an example and not by way of limitation, the prediction of genealogical ancestry can allow for the workflow 100 to provide a family tree 120. In some embodiments, k-means clustering 122 (discussed in more detail below) can be applied to the smoothed local ancestry 116 in order to generate the family tree 120 (or other genealogical information) of the animal.

In certain non-limiting embodiments, the predictor of traits suite can use the global ancestry 118 to generate trait predictions or estimates for animals based on certain genetic probabilities. The global ancestry 118 can be used as an input for a meta-classifier 124, which can provide a whole sample subpopulation label. This meta-classifier 124 can identify one or more predicted classes/groups and confidences 126 for the input global ancestry 118. These classes/groups with confidences 126 can be further used (either alone or in combination with additional genotypes) in various downstream applications 128, which can include predicting lifespan of the subject, genetic dispositions, and other traits inherent in their genome. In some embodiments, the downstream applications 128 can take additional genotypes 130 as input.

These downstream applications 128 can also be used to improve consumer experience 132, allowing for the creation of an application or other service which provides the predictions to an end user.

In certain non-limiting embodiments, the automated system for accuracy improvement can be associated with new reference samples 134, isolation forest outlier detection 136, and cross-validation 138. The automated system can evaluate new reference samples 134 which are added to the reference/training panel 108. This evaluation can include firstly performing a cross-validation 138 across all samples in a candidate reference panel. The cross-validation results can then be used as input to a detection algorithm, for example, an isolation forest outlier detection algorithm 136. As an example and not by way of limitation, based on new reference samples 134a, cross-validation 138a, isolation forest outlier detection 136a, and training panel 108b, the automated system can improve the accuracy for the PBWT matching 110. As another example and not by way of limitation, based on new reference samples 134b, cross-validation 138b, isolation forest outlier detection 136b, and training panel 108d, the automated system can improve the accuracy for the meta-classifier 124.

Local Ancestry Classifier

Conventional local ancestry classifiers can have important limitations. As an example and not by way of limitation, they cannot readily scale to accommodate large reference panels and they can require a large amount of computational resources to produce predictions. By control, the local ancestry classifier as disclosed herein can have improved accuracy over the conventional ones and can readily accommodate much larger reference panels. In particular embodiments, the local ancestry classifier as disclosed herein can use the positional Burrows-Wheeler transform (PBWT) algorithm in conjunction with a mathematical approximation to a standard local ancestry model. In certain non-limiting embodiments, the standard local ancestry model can comprise “chromosome painting.” As used herein, chromosome painting describes a range of techniques that characterize chromosomal rearrangements, including but not limited to employing fluorescently labeled DNA probes. In addition, the local ancestry classifier as disclosed herein can leverage a reference panel to learn common misclassification to smooth the resulting assignments to improve overall accuracy. In some embodiments, the local ancestry classifier can reference a list or matrix comprising common misclassification results in order to smooth the resulting classification. The smoothing can remove commonly mistaken sequences and replace them with their much more likely replacement. The degree to which local ancestry assignments are smoothed can be tuned to accommodate both single origin chromosomes and highly admixed chromosomes. As an example and not by way of limitation, the smoothing can be tuned to accommodate single-origin chromosomes or, alternatively, highly admixes chromosomes, which contain DNA from a plurality of sources.

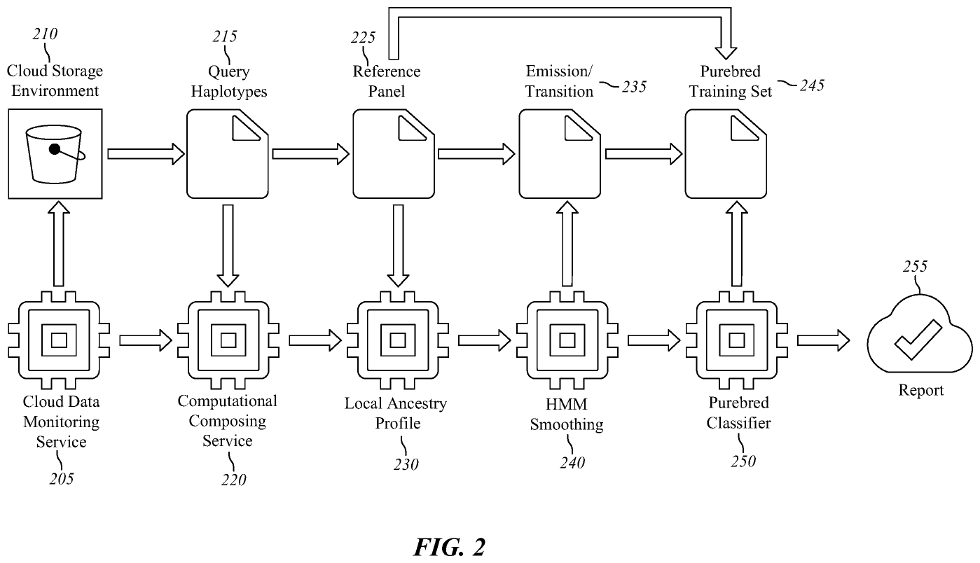

FIG. 2 illustrates an example workflow 200 of the local ancestry classifier. In certain non-limiting embodiments, a cloud data monitoring service 205 can regularly probe a cloud storage environment 210 for the presence of new query DNA sequences. The cloud storage environment 210 can be a scalable storage infrastructure. As an example and not by way of limitation, the query DNA sequence can comprise a plurality of genotype data organized into a plurality of haplotypes 215. The cloud data monitoring service 205 can retrieve the sequences on detection of a positive signal and deposit the query batch in a high-performance compute environment. A computational composing service 220 can then characterize the ingested batch of DNA sequences and compose a custom bioinformatic workflow. The computational composing service 220 can compare the query haplotypes 215 to a reference panel 225 of haplotypes in order to generate a local ancestry profile 230. Emission/transition 235 can be generated based on the reference panel 225 of haplotypes. In some embodiments, the local ancestry profile 230 and the emission/transition 225 can then be smoothed based on HMM smoothing 240 in order to eliminate common errors. In some embodiments, the reference panel 225 can be used as part of a purebred training set 245, based on which a purebred classifier 250 can be learned. Once smoothing is complete, the smoothed local ancestry profile can be processed by the purebred classifier 250 in order to generate a purebred meta-classifier label. Finally, the labeled local ancestry profile can be output to a report 255. As an example and not by way of limitation, the report 255 can be in a JavaScript Object Notation (JSON) format.

In certain non-limiting embodiments, the local ancestry classifier can predict a local ancestry label for a subject. The local ancestry classifier can select two samples, a first sample which corresponds to a query nucleotide sequence and a second sample which corresponds with a reference nucleotide sequence. The query nucleotide sequence can include one or more unknown ancestry labels, wherein the labels can be selected from an ordered set of subpopulation labels. The reference nucleotide sequence can include one or more known genetic subpopulations, which correspond with known nucleotide sequences. Each of the first sample and the second sample can be further partitioned into subregions, also known as windows, to be used in comparing the two samples. At least one subregion of the first sample can then be compared to at least one subregion of the second sample, and nucleotide matches be identified between the two samples. In this way, the degree of similarity between the samples can be determined, by counting the number of nucleotide matches between the first sample and the second sample. A genetic subpopulation, corresponding to and comprising one or more of the nucleotide matches, can be selected from a known listing of genetic subpopulation information. Based on the selected genetic subpopulation, a local ancestry label can be applied, and optionally applied to the one or more query nucleotide sequences.

The identification of a nucleotide match, in any of the exemplary methods, can comprise a variety of different factors, and is not meant to restrict a nucleotide match to an exact match between all elements of the two subregions being compared. For example, in certain non-limiting embodiments, the one or more nucleotide matches can comprise at least one nucleotide sequence within the first sample which is identical to at least one nucleotide sequence within the second sample. In alternative embodiments, a nucleotide match can be determined where at least one nucleotide sequence within the first sample is a predetermined percentage identical to at least one nucleotide sequence within the second sample. Further, in non-limiting embodiments, each of the one or more nucleotide matches can comprise multiple nucleotides. In such embodiments, each of the multiple nucleotides can be identical between the first and the second sample, or, alternatively, can each meet a predetermine percentage of identity between the first and the second sample. In further non-limiting embodiments, the nucleotide matches can comprise adjacent nucleotides.

The number of nucleotide matches can be determined according to a variety of methods. For example, the method can use a length of the number of adjacent nucleotides within the first sample (or a subregion of the first sample) which matches a number of adjacent nucleotides in the second sample (or a subregion of the second sample) in order to calculate the number of nucleotide matches. According to this non-limiting embodiment, the length of the number of adjacent nucleotides in at least one subregion of the first sample and/or the length of number of adjacent nucleotides in the at least one subregion of the second sample can be an approximate length or an exact length.

In non-limiting embodiments, the at least one genetic subpopulation can be determined by examining the nucleotide matches. For example, a genetic subpopulation can be chosen based on the greatest number of nucleotide matches, a specified number of nucleotide matches, and/or a preselected number of nucleotide matches. In further embodiments, the subpopulation can be chosen where the number of nucleotide matches exceeds a particular value or falls within a particular range. The local ancestry classifier can further identify certain outliers relative to a population, and/or removing outliers from a population.

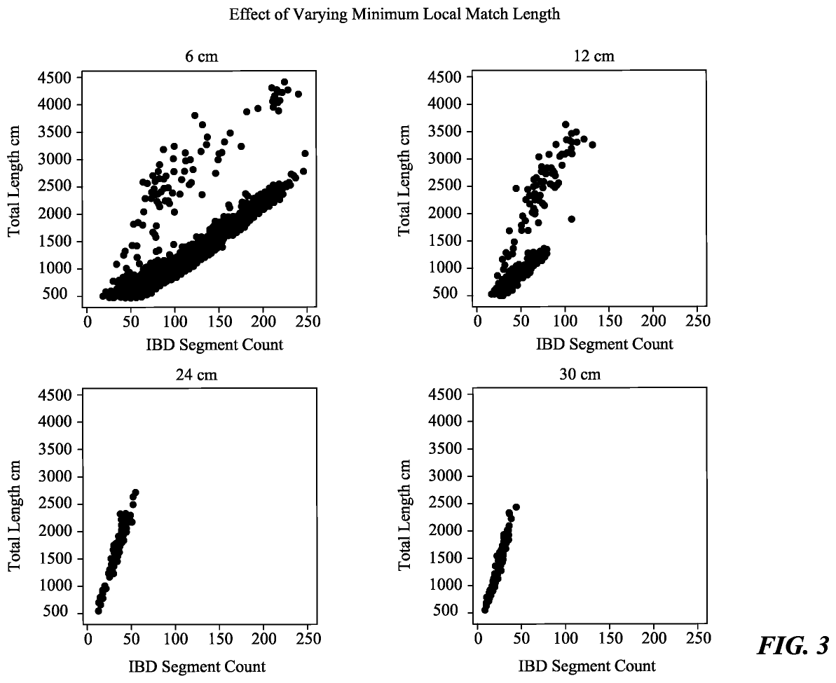

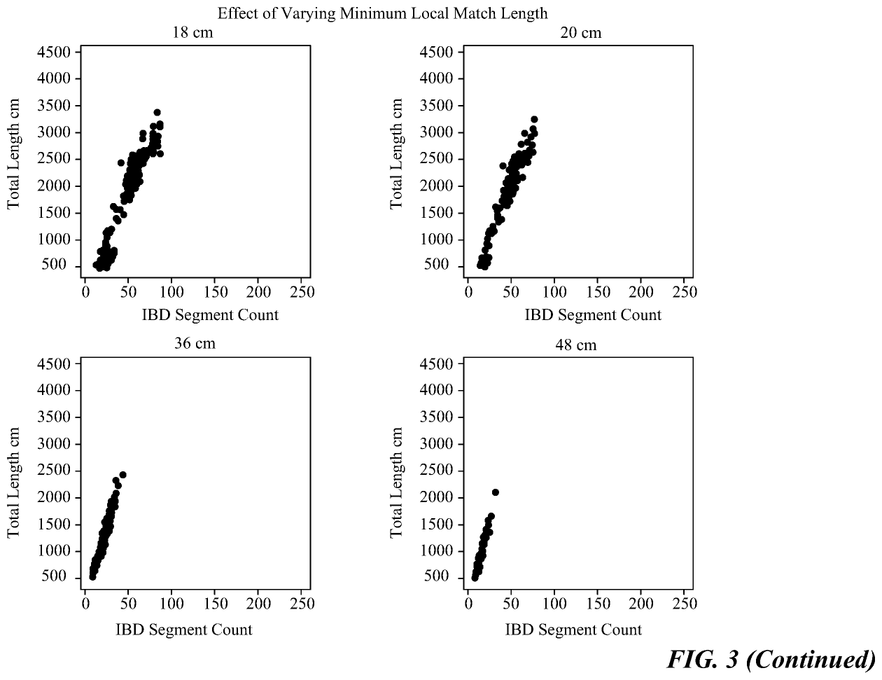

In certain non-limiting embodiments, the local ancestry classifier can assume the existence of a curated reference panel comprising some number of haplotype sequences, each of which can be labelled according to membership in some population group. The goal of the local ancestry classifier can include classifying an arbitrary query haplotype to one of the reference panel populations. The local ancestry classifier can begin by phasing both query and reference genotypes into maternal and paternal chromosomes. In certain non-limiting embodiments, phasing of maternal and paternal genomes can be performed using a phasing reference panel. Alternatively, for example, a phasing reference panel can be obtained by first performing cohort-phasing using the local ancestry reference panel. This phased set of haplotypes is then subsequently used as a panel for reference-based phasing. The phased data can be then partitioned into 5 centimorgan (cM) windows. As an example and not by way of limitation, a window size of 5 cM can be chosen to balance linkage disequilibrium in canids with the recovery of sufficient haplotype diversity to be informative. Although 5 cM windows can be used in certain embodiments, windows of other lengths are considered. FIG. 3 illustrates a plurality of models showing the results of varying the predetermined subregion length, between about 6 centimorgans and about 48 centimorgans. As shown in FIG. 2, windows of lengths 6 cM, 12 cM, 18 cM, 20 cM, 24 cM, 30 cM, 36 cM, and 48 cM can also be used. Further, windows of lengths less than 5 cM can be used, for example, to provide a more detailed view of subregions of the target chromosomes. For the purpose of the presently disclosed subject matter, the length of a window can correspond with the length of any subregion of the first sample or the second sample.

In certain non-limiting embodiments, the population assignment of each window can be achieved by recovering all pairwise set-maximal matches between query and reference haplotypes using a positional Burrows-Wheeler transform algorithm. The density of set-maximal matches between a given query and all reference haplotypes can be calculated and the reference population with the highest relative density can be selected as the “raw” assignment. Then a hidden Markov model (HMM) can be run on the raw calls over windows grouped by chromosome to “smooth” the local ancestry assignments. Finally, the global ancestry proportions can be aggregated from the local assignments and used in the global ancestry classifier to produce a population assignment for the entire diploid genome.

In certain non-limiting embodiments, the local ancestry classifier can recover short matching DNA segments. The set of algorithms inherent in the PBWT can efficiently recover matches between pairs of haplotype sequences in a collection. Several PBWT-based algorithms can iterate through a collection of haplotype sequences and recover set-maximal matches, which can be defined as the set of other sequences which show locally maximal, unbroken matches to the current sequence. In the disclosure presented herein, the collection of sequences can comprise both query and reference haplotypes.

As described above, the PBWT can include a collection of related algorithms for fast sorting of binary matrices. The algorithm can operate on a binary matrix that has N rows representing haplotypes and M columns representing biallelic DNA sites. Rows can be sorted sequentially, starting from the leftmost column. As the algorithm proceeds, site by site, two vectors can be updated: the first is the rank order of the haplotypes (positional prefix array) and the second is a measure of the number of differences with the immediately preceding haplotype (divergence array). Elements of the divergence array can be additive across ordered haplotypes, resulting in a Hamming distance between haplotypes. By keeping track of which sequences are adjacent in the positional prefix array and whether the divergence array is zero, matching haplotype sequences can be retrieved. Matches can be broken when the haplotypes are no longer adjacent or the corresponding element of the divergence array is no longer zero. In certain non-limiting embodiments, a set-maximal match can be a locally maximal match to a given sequence (over the interval ending at the current position) and can include one or more adjacent haplotypes that have the longest match over that interval.

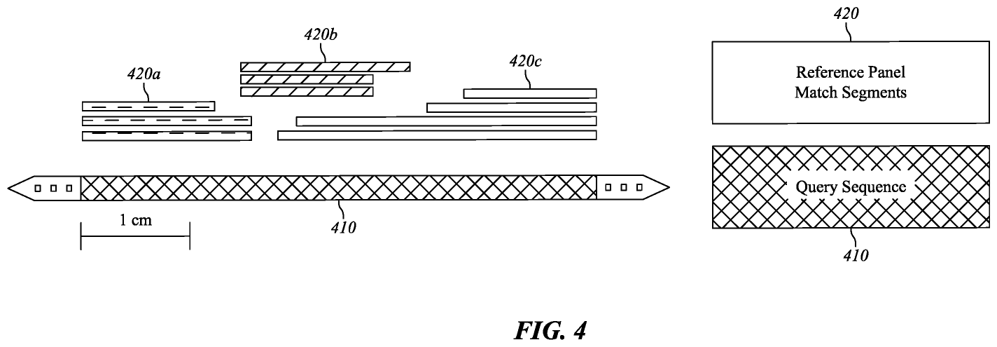

FIG. 4 illustrates an example marginal match length according to the presently disclosed subject matter. The query sequence 410 is depicted at the bottom. Matches to reference panel sequences 420 are shown above. Matches 420a to matches 420c can correspond to its corresponding reference population label, respectively. The marginal match length sum per reference population can be considered proportional to the likelihood of the query sequence originating from that reference population.

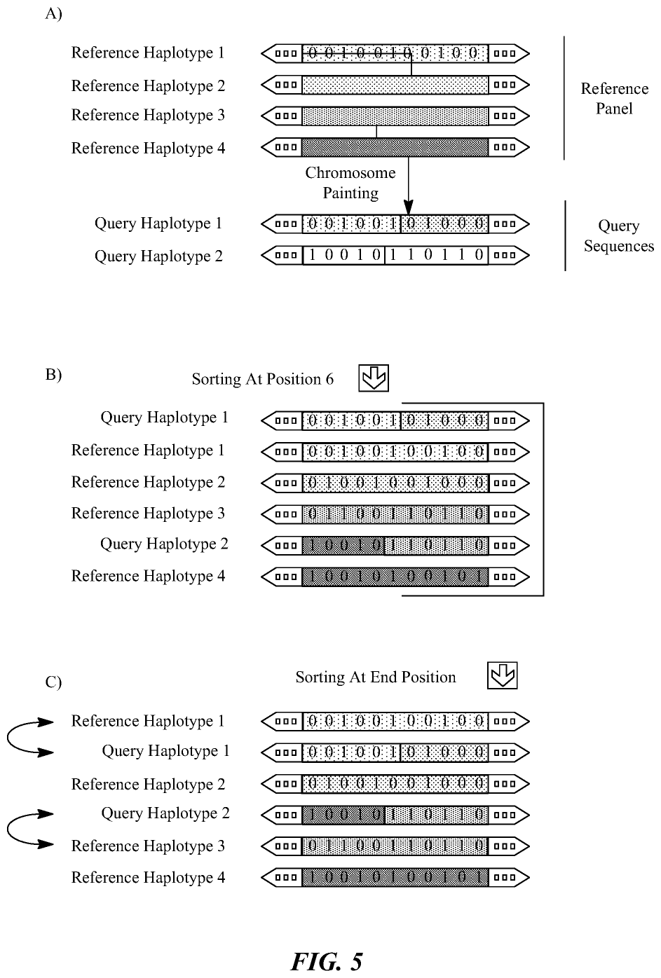

FIG. 5 illustrates an example comparison between a “chromosome painting” model (A) and the PBWT-based model described in this disclosure (B and C). In panel A, the query sequences are compared against all reference panel sequences and the most likely path through the reference panel sequences can be responsible for labelling or “painting” the query chromosomes. In the PBWT-based method, sequences can be progressively sorted in such a way that locally matching sequences are adjacent in the list. For example, in panel B, the PBWT algorithm has sorted up to position 6 and in panel C, the algorithm has sorted up to the final position. The query chromosomes can be “painted” by evaluating which sequences are adjacent in the PBWT data structure. This simplification allows the PBWT-based method to scale more readily to very large reference panel sizes. As indicated in FIG. 4, the PBWT can be chosen to sort at a particular position among the selected query genotype sequences, for example, at position 6 within the query genotype and, as an alternative example, at the end position of the query genotype. Alternative methods of achieving a population assignment can be used, for example, chromosome painting. The density of set-maximal matches between a given query and all reference haplotypes can be calculated and the reference subpopulation with the highest relative density can be selected as the raw assignment. This density of matches can also correspond to the number of nucleotide matches between selected samples.

In certain non-limiting embodiments, the local ancestry classifier can assume the availability of a curated population reference panel comprising a total of N phased haplotypes. Each reference haplotype can be assigned a single label from k, which is an ordered set of K source population labels. Furthermore, there can be a corresponding ordered set n of subpopulation sample sizes, such that N=Σᵢ₌₁^{K} nᵢ.

In addition to the reference panel haplotypes, the local ancestry classifier can consider a single query haplotype which will be assigned a label from k. After running the PBWT-based algorithm described above, all set-maximal matches can be recovered between the query haplotype and the reference panel haplotypes. Each set-maximal match can be labeled by the reference population label of the matching haplotype (see FIG. 4). To exclude small haplotype segments with high homozygosity across source populations (and therefore are unlikely to arise due to recent common ancestry), set-maximal matches longer than 0.5 cM can be considered in the analysis. Each of the recovered set-maximal match lengths can be labelled by the corresponding source population label in k. The marginal sum of match lengths with label i is denoted lᵢ.

In certain non-limiting embodiments, the local ancestry classifier can determine the probability that the query haplotype is sampled from source population i. These probabilities can constitute an ordered set of source population sampling probabilities p, which parameterize a categorical distribution with probability mass function P(Q=i|p)=pᵢ, wherein Q is the source population label of the query haplotype. The query haplotype can be assigned the label Q=kᵢ according to the criterion maxᵢ pᵢ.

In certain non-limiting embodiments, the marginal match lengths described above can formulate a statistic to estimate p. The marginal match lengths can be drawn from a sample space encompassing the total length of all source population haplotypes, Lᵢ=Lᵢₙₜ×nᵢ, where Lᵢₙₜ is the recombination distance of the genomic interval under consideration. A first-moment statistic can be defined as follows:

$${g}_{i} = \frac{{ℓ}_{i}}{{L}_{i}} .$$

This statistic can estimate the proportion of all source population haplotypes matching the query and, in practice, it can be expected that gᵢ<<1.

The parameters of the categorical distribution can be approximated by standardizing the statistic:

$${p}_{i} ∝ \frac{{g}_{i}}{{\sum }_{i}^{K} {g}_{i}} .$$

In contrast to other local ancestry algorithms, the local ancestry classifier disclosed herein can be a simple moment-based estimator, which minimizes reliance on complex underlying population genetics models that are often needed when a Dirichlet distribution is used as a conjugate prior for the categorical distribution. Bayesian inference under a Dirichlet prior can necessitate assumptions inherent in the simulation of highly stochastic population structure models with uncharacterized parameters, at the expense of scalability and increased computation time, often with an unknown improvement in accuracy. Rather than leveraging traditional simulation-based Bayesian inference, the local ancestry classifier disclosed herein instead focuses on improving assignment accuracy through application of machine learning models trained on reference panel samples.

In certain non-limiting embodiments, the method of using marginal reference source population match lengths can also lend increased robustness to haplotype phasing errors present in the reference panel haplotypes. The rationale can be that long matches broken by phase switches can still be recovered as separate matches by the algorithm and contribute equivalently to the marginal sum of match lengths. The scenario for which this can be not the case is when a phase switch breaks a long match and one (or both) of the resulting match segments are too short (i.e., <0.5 cM) to be recorded by the method. In some embodiments, the estimate of the marginal population match length can be reduced by a maximum of 1 cM. One approach for addressing this case can be to reduce the match length threshold.

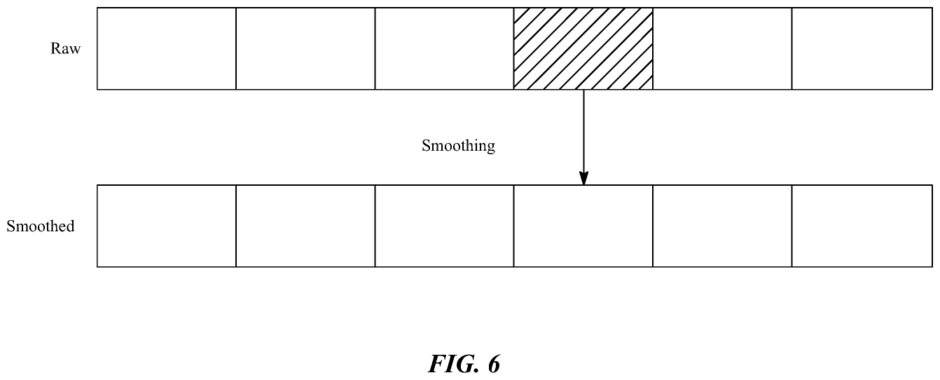

In certain non-limiting embodiments, the local ancestry predictions can be smoothed. FIG. 6 illustrates an example smoothing process. As shown in FIG. 6, the raw assignment data can be further smoothed to remove common errors or improve accuracy. For example, in certain non-limiting embodiments, a machine learning model can be run on the raw calls over all windows in the raw assignment data set to smooth the local ancestry estimates. A variety of machine learning models can be used, including, but not limited to, hidden Markov models. The smoothing can also obtain global subpopulation proportions (i.e., global ancestry) from the local estimates and these global estimates can be used in conjunction with a plurality of meta-classifiers to produce a whole sample subpopulation label and local ancestry calls can be partitioned as being either maternally or paternally inherited.

Hidden Markov models (HMM) are widely used in population genomics because they model the linear nature of features along chromosomes. In the disclosure presented herein, the ordered sequence of local ancestry labels can be treated as the observed sequence in an HMM. In this framework, each reference population can be considered a latent variable, or a “hidden state” of the query haplotype. The objective of employing the HMM in this manner can be to eliminate spurious transitions between local ancestry assignments and to correct for common mis-assignments. In certain non-limiting embodiments, the local ancestry classifier can favor HMM parameters to encourage mixing of the chain, such as adding pseudo counts to transition probabilities to ensure no probability is zero so that the local ancestry classifier can perform well for highly admixed samples. In some embodiments, the HMM can be trained on the reference panel, for which the local ancestry assignments are assumed to be a source of truth.

The HMM emission probabilities can be estimated by a leave-one-out procedure applied to all reference panel haplotypes. Each of the reference haplotypes is used as a query sequence and assigned subpopulation labels from the estimated parameters of the categorical distribution p. These estimates can be aggregated over all N of the query haplotype runs into a K×K matrix binned by the “true” subpopulation label of the haplotype. The elements of the resulting population confusion matrix can be used as the HMM emission probabilities. The transition matrix can be also learned from the estimated sequence of population labels in the reference panel haplotypes. Finally, the vector of probabilities of starting in a given hidden state can be estimated from the global ancestry estimates resulting from the PBWT-based calls. A separate HMM can be run for each chromosome using the backward-forward algorithm, and the most likely pathway through the hidden states can be decoded using the Viterbi algorithm.

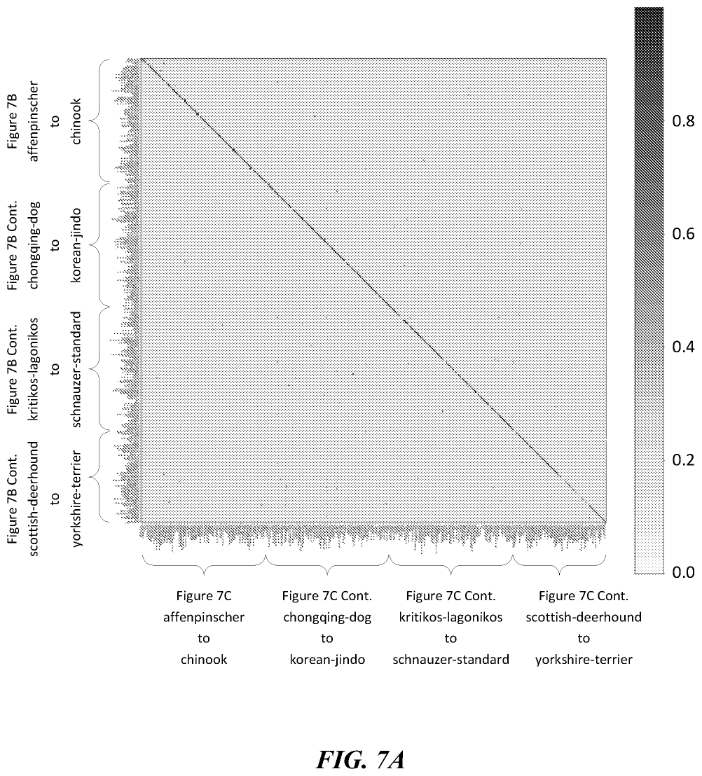

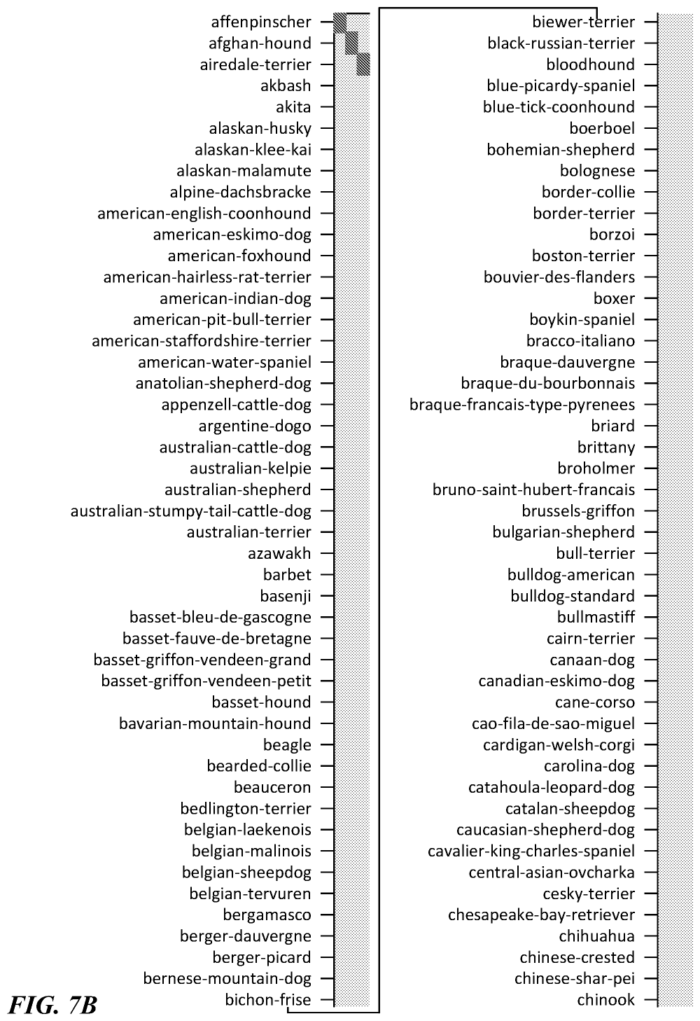

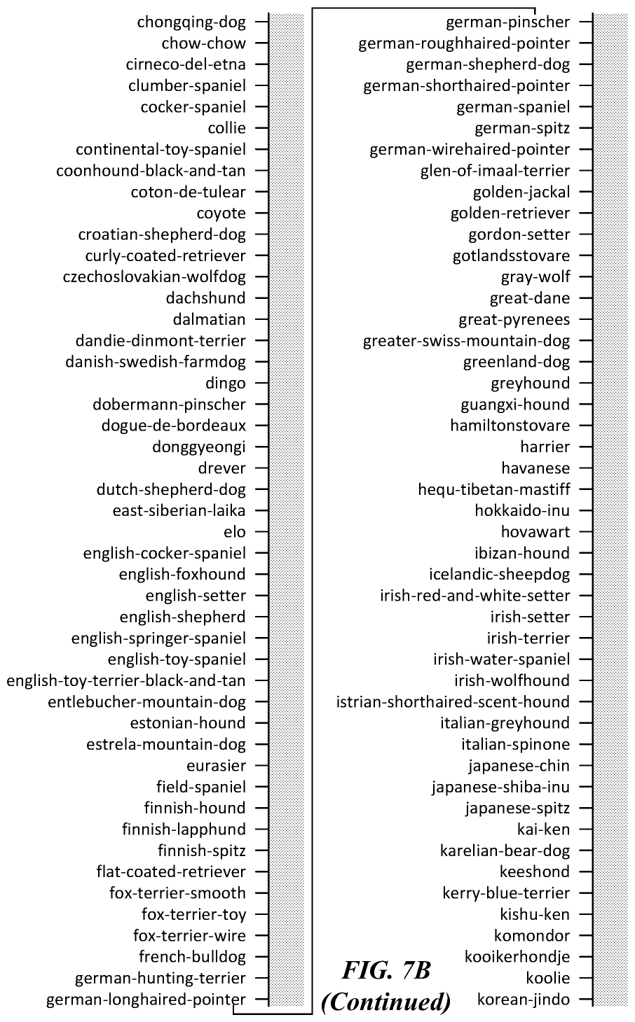

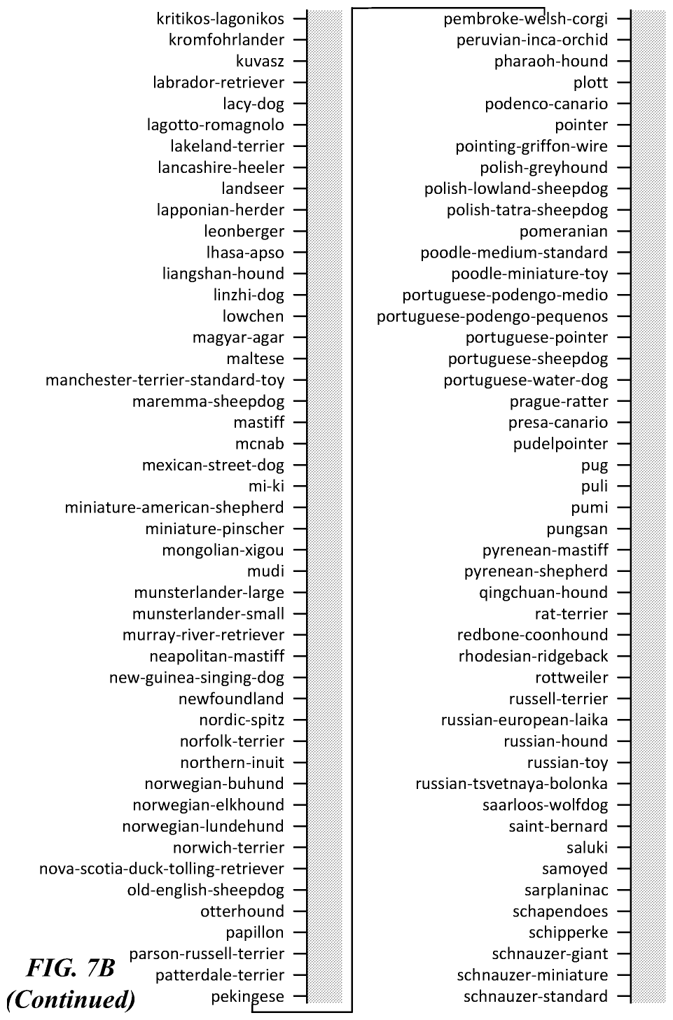

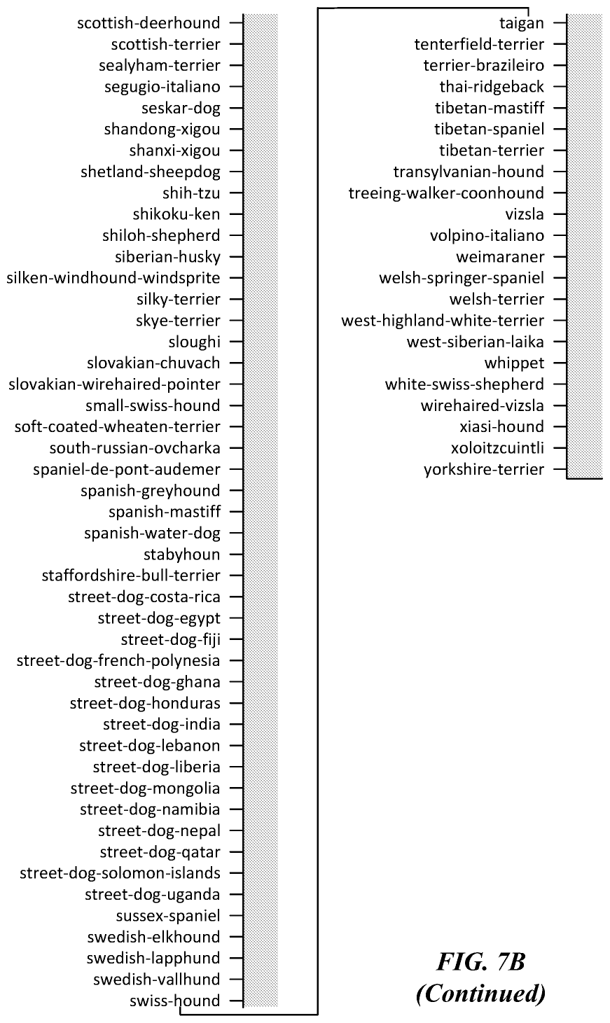

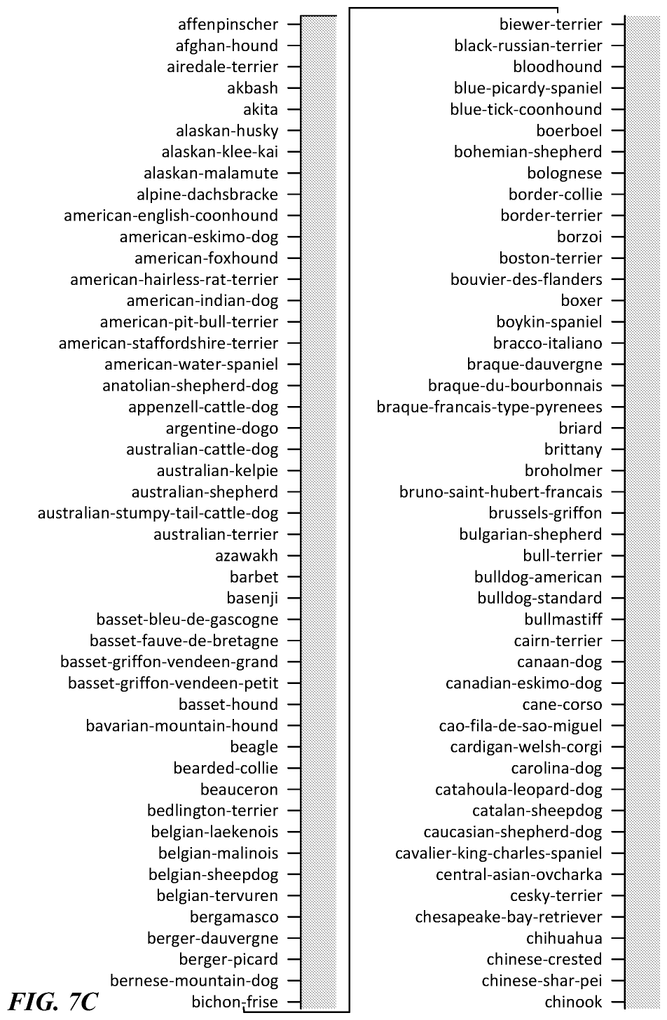

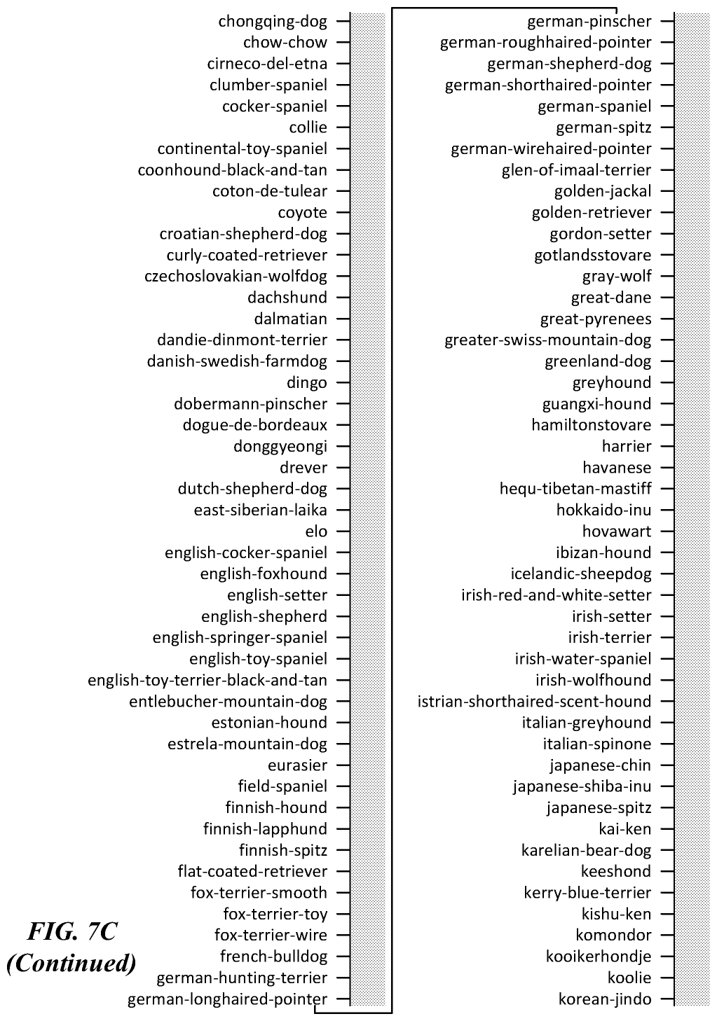

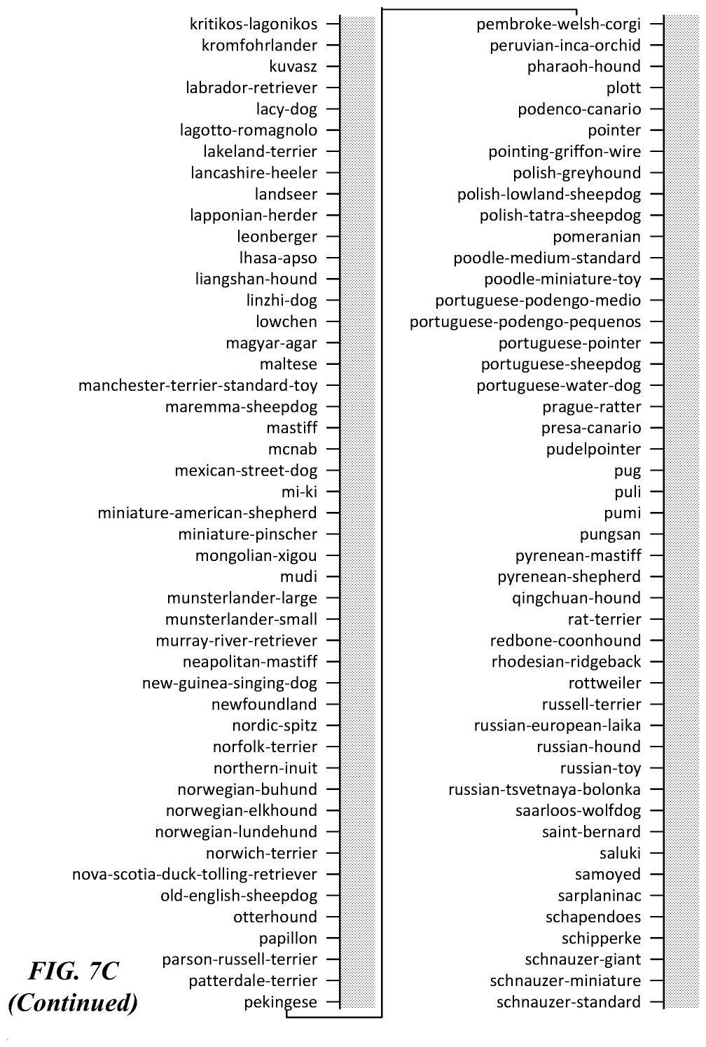

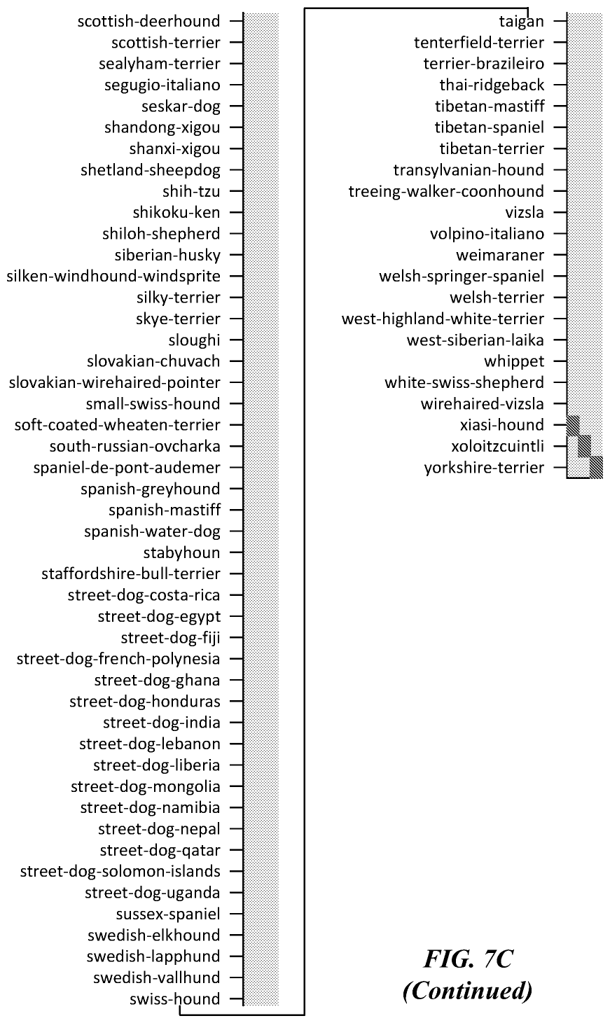

In certain non-limiting embodiments, the smoothing method can comprise a plurality of steps. As an example and not by way of limitation, the method can identify a first portion of at least one of the two or more subregions of the first sample of genetic material. The method can then identify a second portion of at least one of the two or more subregions of the first sample of genetic materials. The method can then replace the second portion with the first portion. The smoothing method can be performed where the second portion is one that is commonly confused with the first portion, for example, where identification of the second portion of the subregion as a particular breed is a common error, with the first portion representing the correct breed. The smoothing method can help to improve accuracy of the overall workflow and results in a more accurate breed identification. In some embodiments, identification of commonly confused breeds and/or species can be facilitated by a confusion matrix. FIG. 7A illustrates a confusion matrix related to a plurality of animal species and/or of animal breeds. FIG. 7B shows animal breeds of the y-axis. FIG. 7C shows animal breed of the x-axis. FIGS. 7A-7C show that the confusion matrix can be useful for identification of breeds and/or species.

Due to the techniques utilized by the local ancestry classifier as described above, the local ancestry classifier can have improved accuracy over the conventional work and can readily accommodate much larger reference panels than the conventional work. Such advantage will be described in the section of “Examples”, specifically “Benchmarking Accuracy of Ancestry Classifiers” and “Benchmarking Scalability of Classification System” later in this disclosure.

Global Ancestry Classifier

In certain non-limiting embodiments, the global ancestry classifier can consider the totality of local ancestry classifications to predict the source population(s) for the entire organism. This can include organisms that originate from a single source population but can also include commonly seen combinations (or admixtures) of source populations. With regard to companion animals, a straightforward example can be predicting a “goldendoodle”, which is a cross between a golden retriever and a poodle. In addition, the global ancestry classifier can upweight specific DNA variants known to influence particular traits, in order to refine predictions for source populations that can be otherwise indistinguishable at the whole-genome level. For example, a variant of the fibroblast growth factor gene FGF5 is known to influence coat length in domestic dogs. For some dog breeds with varieties with different coat length, which would otherwise be indistinguishable across the whole genome, upweighting the FGF5 gene variant can accurately distinguish long-haired versus short-haired varieties.

In certain non-limiting embodiments, the local ancestry assignments from the Viterbi path can be aggregated over both maternal and paternal chromosome sets and used to calculate the global ancestry proportions for a given diploid sample. The global ancestry proportions can be used as features to predict the population label for the entire diploid sample using a Random Forest classifier. The predictions can be associated with confidence scores recalibrated by one or more algorithms. In some embodiments, the Random Forest classifier can be trained on the reference panel leave-one-out results described above (after being run through an HMM).

Due to the techniques utilized by the global ancestry classifier as described above, the global ancestry classifier can have advantageous functionality and performance than conventional work. Such advantage will be described in the section of “Examples”, specifically “Benchmarking Accuracy of Ancestry Classifiers”, “Benchmarking Scalability of Classification System”, and “Assessing Accuracy of Global Ancestry Classifier” later in this disclosure.

Predictor Of Genealogical Ancestry

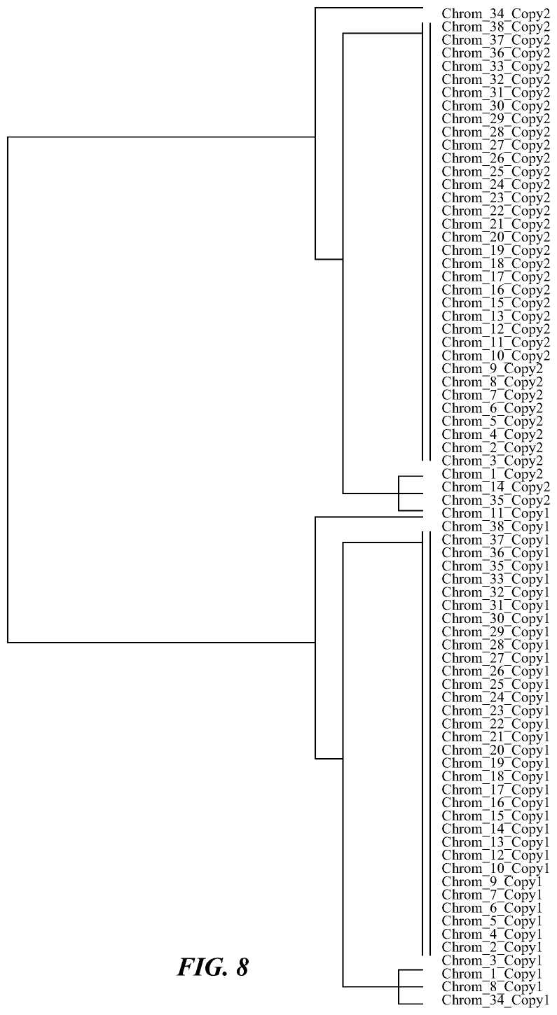

Since local ancestry methods predict the source population(s) for a single phased copy of a chromosome, the local ancestry predictions can be further partitioned into maternally and paternally inherited. In certain non-limiting embodiments, the predictor of genealogical ancestry for partitioning parental chromosomes can assume that the local ancestry proportions making up a single haploid copy of the genome are similar across different chromosomes. The predictor of genealogical ancestry can then find the most likely partition of maternal and paternal chromosomes by minimizing the Euclidean distance between a full complement of haploid chromosomes. FIG. 8 illustrates an example sort of chromosome pairs into maternal and paternal copies using k-means clustering. In FIG. 8, example of partitioning of 38 canid chromosome pairs into maternal and paternal copies using k-means clustering based on chromosome-specific local ancestry proportions.

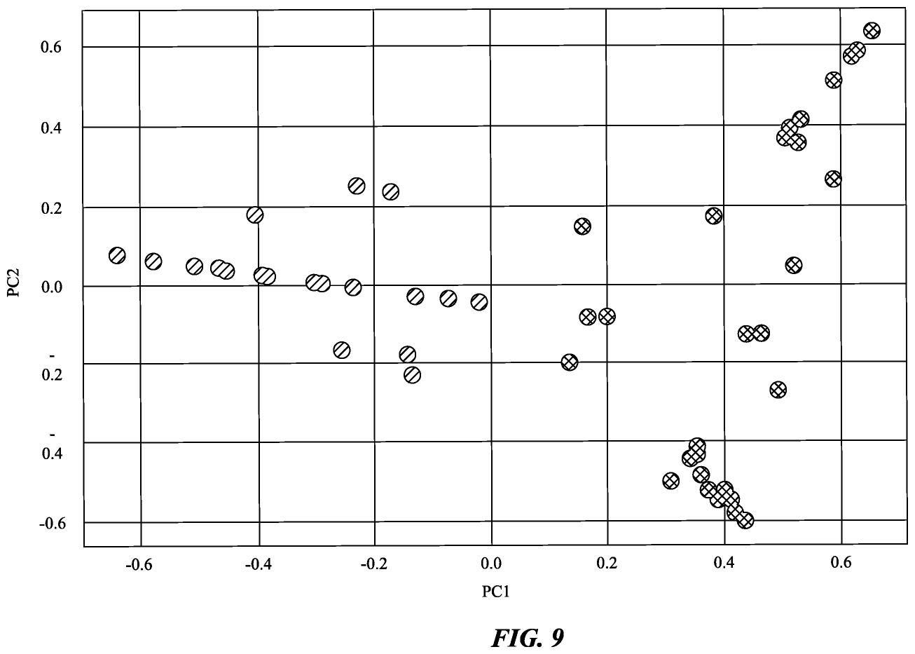

In certain non-limiting embodiments, the predictor of genealogical ancestry can use eigen-decomposition of a matrix of per-chromosome global ancestry proportions. The rows of the matrix can be haploid chromosomes and columns can be the source population labels. The two resulting components can be subject to k-means clustering with k=2 (arbitrarily maternal and paternal groupings). The aim can be to group chromosomes by similar ancestry composition and use this criterion to partition each chromosome into a maternal and paternal set. This procedure can serve as the basis for reconstituting a genealogical history of an individual companion animal FIG. 9 illustrates example principal components from global ancestry proportions for a set of chromosomes. FIG. 9 shows the plot of first two principal components from global ancestry proportions for each of 38 pairs of canid chromosomes. Parental chromosome sets are arbitrarily labelled as maternally or paternally inherited.

Due to the techniques utilized by the predictor of genealogical ancestry as described above, the predictor of genealogical ancestry can further partition the local ancestry predictions into maternally and paternally inherited, which can be a unique feature.

Predictor of Traits Suite

In certain non-limiting embodiments, the output from local ancestry classifier and/or global ancestry classifier can be used as input for the predictor of traits suite comprising a series of trait prediction modules. These prediction modules can take a variety of auxiliary input, including genotypes of variants of large effect, genome-wide statistics (e.g., average homozygosity), genomic principal component analysis (PCA) projections, DNA methylation profiles, and/or polygenic risk scores. As an example and not by way of limitation, the predictor of traits suite can predict one or more of expected healthy adult body weight with range prediction, risk prediction and predisposition to genetic disease, nutrition recommendations based on ancestry classification, behavior and temperament class prediction, longevity and all-causes mortality prediction in years, or predicted pharmacological response, recovery time range in hours for injectable anesthetics. In some embodiments, the nutrition recommendations can include a recommendation of one or more pet food products comprising a commercially-available pet food product and/or an individualized pet food product.

In certain non-limiting embodiments, the predictor of traits suite can use the local ancestry classification to determine certain predictions or estimations of various characteristics of the subject, by using, for example, the local ancestry label to identify known gene sequences which contribute to certain traits. As an example and not by way of limitation, the predictor of traits suite can use the local ancestry label to identify one or more ranges of the adult body weight of the subject; to identify one or more predisposition to one or more genetic diseases; to provide one or more nutrition product recommendations and/or one or more nutrition regimen recommendations; to estimate longevity and/or lifespan for the subject; and/or to predict one or more pharmacological responses for the subject.

As described above, by utilizing the input from the local and global ancestry classifiers and various auxiliary input, the predictor of traits suite can predict much more traits than conventional work. Such advantage will be described in the section of “Examples”, specifically “Performance of Trait Prediction” later in this disclosure.

Automated System for Accuracy Improvement

The accuracy of produced classifiers can depend on the individual samples in a source population reference panel. As an example and not by way of limitation, where the source population reference panel contains incorrect population labels, accuracy of the entire workflow 100 of the system can be reduced. In certain non-limiting embodiments, the automated system for accuracy improvement can evaluate new samples which are added to the reference panel. This evaluation can include firstly performing a cross-validation by a leave-one-out method across all samples in a candidate reference panel. The cross-validation results can then be used as input to a detection algorithm, for example, an isolation forest anomaly detection algorithm. The algorithm can identify certain samples as outliers, relative to their population labels, and remove those samples from the reference panel. The automated system can run repeatedly as appropriate, until a predetermined level of accuracy is reached, for example, until panel precision and recall cease to improve significantly. In alternative non-limiting embodiments, a machine learning algorithm can be used to generate labels for unlabeled samples. As an example and not by way of limitation, a semi-supervised machine learning label propagation algorithm can be used to automate the assignment of putative labels to unlabeled samples.

As described above, the automated system for accuracy improvement can utilize cross-validation of the reference panel by a leave-one-out procedure. In this scenario, each sample included in the reference panel can be iteratively removed from the panel and can be then run as a query sequence. The left-out query sequence can be then assigned local ancestry labels. This procedure can be repeated for all samples included in the reference panel. Samples can be then grouped by putative source population labels. The isolation forest technique can be run on each set of samples grouped by source population label, using the local ancestry calls as features. The number of tree partitions induced to isolate a given sample can be used as a decision function to identify anomalies. When a forest of random trees produces shorter than expected path lengths for a particular sample, that sample can be labelled as an anomaly and can be removed from the reference panel. This procedure can be repeated until the improvements in weighted recall and precision fall below a pre-specified threshold value.

Due to the techniques utilized by the automated system for accuracy improvement as described above, the automated system can further improve the performance of the system and subsystems as disclosed herein. Such advantage will be described in the section of “Examples”, specifically “Performance of Automated Accuracy Improvement” later in this disclosure.

### 3. SEQUENCING, KITS, AND METHODS OF TREATMENT

The present disclosure includes methods for sequencing the genome of an animal or a pet. The terms “animal” or “pet,” as used in, accordance with the present disclosure refer to domestic animals including, but not limited to, domestic dogs, domestic cats, horses, cows, ferrets, rabbits, pigs, rats, mice, gerbils, hamsters, goats, and the like. Domestic dogs and cats are particular non-limiting examples of pets. The term “animal” or “pet” as used in accordance with the present disclosure can further refer to wild animals, including, but not limited to bison, elk, deer, venison, duck, fowl, fish, and the like.

As used herein, the terms “dog” or “canine” are used interchangeably and refer to any member of the Canidae family including, but not limited to, Canis lupus, Canis familiaris, Canis latrans, Canis dingo, Lycaon pictus, Chrysocyon brachyurus, Atelocynus microns, Cuon alpinus, Speothos venaticus, Nyctereutes procyonoides, Vulpes vulpes, and Alopex lagopus. In certain embodiments, the dog or canine is Canis familiaris.

In certain embodiments, the method comprises obtaining a sample from the animal. In certain embodiments, the sample can be a bodily fluid obtained from the animal. In certain non-limiting embodiments, the sample can be saliva, sputum, blood, perspiratory fluid (e.g., sweat), pus, tear, mucosal excretion, vomit, urine, stool, semen, vaginal fluids, or other types of bodily fluid. In certain embodiments, the sample can be a non-fluid sample. In certain embodiments, the sample can be a cell-free sample. For example, but without any limitation, the sample is a cell-free nucleic acid sample. In certain embodiments, the sample can include cell-free deoxyribonucleic acid (DNA), cell-free ribonucleic acid (RNA), and/or cell-free protein. In certain embodiments, the sample can include one or more cells.

In certain embodiments, the sample can be a solid or tissue sample. In certain embodiments, the sample can be a skin sample. In certain embodiments, the sample can be a cheek swab or a swab of a different bodily part. In certain embodiments, the sample can be a homogenous sample or a heterogeneous sample. In certain embodiments, the sample can be a tumor sample. In certain embodiments, the sample can include one or more types of different biological samples. For example, but without any limitation, the sample can include saliva and skin tissue. In certain embodiments, the sample can be a plasma or serum sample.

In certain embodiments, the sample is a sputum sample. In certain embodiments, the sample is a saliva sample. In certain embodiments, the sample is a cheek swab.

In certain embodiments, the sample can be collected from the animal and preserved and/or stabilized until a time of further processing and/or analysis. For example, but without any limitation, the sample can be preserved and/or stabilized by incubation with a reagent for such use. In certain embodiments, the reagent for preserving and/or stabilizing the sample can be any substance acting on the collected sample to achieve a desired effect. In certain embodiments, the reagent can be in any suitable form, such as a fluid (e.g., liquid, gas, solution, etc.) or a non-fluid (e.g., solid powder, etc.). In certain embodiments, the reagent can preserve deoxyribonucleic acid (DNA), ribonucleic acid (RNA), proteins, or other components of proteins in the sample. In certain embodiments, the reagent can prevent alterations in the cellular epigenome of one or more cells. In certain embodiments, the reagent can permit the extraction of a desired molecule (e.g., nucleic acid molecules) from a cell from the collected sample. In certain embodiments, the reagent can be configured to otherwise process the collected sample and/or one or more constituents thereof. In another non-limiting example, the collected sample can be preserved in its original state until further processing and/or analysis. In certain embodiments, the collected sample can be preserved and/or stabilized to prevent bacterial or fungal growth. In certain embodiments, the collected sample can be preserved for at least about 1 hour, about 2 hours, about 3 hours, about 4 hours, about 5 hours, about 6 hours, about 12 hours, about 1 day, about 2 days, about 3 days, about 4 days, about 5 days, about 6 days, about 7 days, about 1 week, about 2 weeks, about 3 weeks, about 4 weeks, about 1 month, about 2 months, about 3 months, about 4 months, about 5 months, about 6 months, about 1 year, about 2 years, about 3 years, or for a longer time. In certain embodiments, the collected sample can be preserved and stored at room temperature or lower for prolonged periods of time. In certain embodiments, the collected sample can be preserved and stored at ambient temperatures or lower for prolonged periods of time. In certain embodiments, the collected sample can be preserved at temperatures of up to about 60° C.

In certain embodiments, the stabilized and/or preserved sample can be further processed and analyzed at an outside facility (e.g., a remote facility). For example, but without any limitation, nucleic acid molecules (e.g., DNA or RNA) from the sample can be isolated and extracted for amplification and/or sequencing applications.

After collecting the sample, the sample can be processed to extract nucleic acid molecules (e.g., DNA or RNA). In certain embodiments, DNA extraction methods include organic extraction (e.g., phenol-chloroform method), nonorganic method (e.g., salting out and proteinase K treatment), and adsorption method (e.g., silica-gel membrane). Additional non-limiting examples of techniques for isolating nucleic acids include the Qiagen DNeasy Kit™ Qiagen QIAamp Cador Pathogen Mini Kit™, the Nucleospin 96 Tissue kit (Macherey-Nagel), QIAzol Lysis Reagent, Qiagen RNeasy kit, Qiagen TurboCapture mRNA kit, and Isopropanol DNA Extraction.

In certain embodiments, the methods disclosed herein comprise detection and quantification of the genome of an animal or a pet. In certain embodiments, the detection and quantification of the genome include isolating DNA from the sample and sequencing the DNA. In certain embodiments, the detection and quantification of the genome include isolating DNA from the sample and quantifying the DNA (e.g., quantitative PCR).

Any suitable technique for detecting and quantifying the genome of an animal or a pet can be employed. Examples of techniques for detecting and quantifying the genome of an animal or a pet include, but are not limited to, 454 pyrosequencing, polymerase chain reaction (PCR), quantitative PCR (qPCR), shotgun sequencing, metagenome sequencing, Illumina sequencing, PacBio sequencing, nanopore sequencing, and microarray genotyping. In certain non-limiting embodiments, the genome of an animal or a pet can be determined by qPCR amplification and sequencing of certain genetic loci. In certain embodiments, the sequencing method is a 454-pyrosequencing. In certain embodiments, the sequencing method is Illumina sequencing. In certain embodiments, the sequencing method is whole-genome sequencing. In certain embodiments, the method for detecting and quantifying the genome of an animal or a pet is microarray genotyping. In certain embodiments, the microarray genotyping is Illumina Infinium BeadChip microarray genotyping.

The genome of an animal or pet can be further analyzed using any of the methods disclosed herein.

In certain embodiments, the present disclosure comprises systems, devices, and methods to allow convenient and simple at-home, on-site, or remote collection of samples. For example, any user can collect a sample without direct supervision. In certain embodiments, the sample can be collected in a sample collection device. In certain embodiments, the sample collection device can include a reservoir pre-loaded with chemical reagents for preserving and/or storing the sample (e.g., nucleic acid molecules). In certain embodiments, the reservoir of the sample collection device can be advantageously shielded from direct exposure to the user. In certain embodiments, the user can be provided with easy-to-follow instructions. In certain embodiments, the instructions can instruct on how to use a device, collect a sample using the device, dispose (e.g., ship to a remote location) of the device after use, access results from analysis of the sample, or other instructions. In certain embodiments, the collected sample can be transported, such as via shipping (e.g., through the mail or a carrier), to a remote lab for further processing and/or analysis.

In certain embodiments, the sample collection device can include a carrier onto which the biological sample will be collected. In certain embodiments, the carrier can be an absorbent member. For example, but without any limitation, the carrier can be a swab, cotton, pad, sponge, foam, or other material or device capable of carrying the biological sample by absorbing.

In certain embodiments, the present disclosure provides a kit. In certain embodiments, the kit includes a sample collection device. In certain embodiments, the sample collection device includes a reservoir and a carrier. In certain embodiments, the reservoir includes a reagent for stabilizing and/or preserving a sample. In certain embodiments, the reservoir includes a shield to protect a user from direct exposure to the reagent. In certain embodiments, the carrier includes an absorbent member. In certain embodiments, the carrier is a swab. In certain embodiments, the reservoir and the carrier are configured and arranged to limit or avoid the spilling of reagents or samples. In certain embodiments, the kit includes written instructions. Written instructions can be provided in a pamphlet or using an internet connection (e.g., using a QR-code). For example, but without any limitations, the instructions can include information on how to use the sample collection device, how to collect the sample, how to dispose, and how to access results from the analysis of the sample.

In certain embodiments, the kit includes a container for shipping the sample collection device to a remote processing location. In certain embodiments, the kit includes boxes, envelopes, or other packaging material (e.g., insulating material, self-sealing or another sealing mechanism, postage, etc.). In certain embodiments, the kit includes a return label and/or a prepaid label.

In certain embodiments, the kit includes instructions on how to access results from the analysis of the sample. In certain embodiments, the instructions can include a hyperlink or a quick-response code (e.g., QR-code) to allow access to a website or download an application on a personal device (e.g., smartphone). In certain embodiments, the results are provided in a report. In certain embodiments, the report is delivered via mail or electronically to a user or a healthcare provider (e.g., a veterinarian). In certain embodiments, the report can be visualized on a personal device (e.g., a smartphone). In certain embodiments, the report can include customized recommendations.

In certain embodiments, the customized recommendation includes administering the animal an individualized nutritionally complete diet. For example, but without any limitations, the customized recommendation could be one diet described in International Patent Publication No. WO 2021/061743, the content of which is incorporated by reference in its entirety.

In certain embodiments, the customized recommendation includes administering a weight gain diet or a weight loss diet. In certain embodiments, the diet (e.g., weight loss diet or weight gain diet) is tailored based on the current weight of the animal and the genome of the animal. In certain non-limiting embodiments, the diet comprises energy density of about 4100 kcal/kg, about 4000 kcal/kg, about 3900 kcal/kg, about 3800 kcal/kg, about 3700 kcal/kg, about 3600 kcal/kg, about 3500 kcal/kg, about 3000 kcal/kg, about 2500 kcal/kg, about 2000 kcal/kg, about 1500 kcal/kg, about 1000 kcal/kg or less, or any intermediate value or range thereof. In certain non-limiting embodiments, the diet comprises an amount of fat of about 20% w/w, 19% w/w, 18% w/w, 17% w/w, 16% w/w, 15% w/w, 14% w/w, 13% w/w, 12% w/w, 11% w/w, 10% w/w, 9% w/w, 8% w/w, 7% w/w, 6% w/w, 5% w/w, 4% w/w, 3% w/w, 2% w/w, 1% w/w or less, or any intermediate value or range thereof. In certain non-limiting embodiments, the diet comprises an amount of carbohydrates is about 25% w/w, 20% w/w, 15% w/w, 10% w/w, 5% w/w, 1% w/w or less, or any intermediate value or range thereof. In certain non-limiting embodiments, the diet comprises an amount of protein is about 20% w/w, 25% w/w, 30% w/w, 35% w/w, 40% w/w, 45% w/w or more, or any intermediate value or range thereof. In certain non-limiting embodiments, the diet comprises an amount of dietary fiber is about 5% w/w, 10% w/w, 15% w/w, 20% w/w, 25% w/w, 30% w/w, 35% w/w, 40% w/w, 45% w/w or more, or any intermediate value or range thereof. Additional information on weight loss diets and weight gain diets can be found in International Patent Publication No. WO 2018/129518, the content of which is incorporated by reference in its entirety.

In certain embodiments, the customized recommendation includes administering the animal a diet to improve skin condition (e.g., hydration, texture, elasticity, integrity, barrier, etc.). In certain embodiments, the diet comprises linoleic acid. In certain embodiments, the diet comprises linoleic acid in an amount from about 7 g/Mcal to about 9 g/Mcal. In certain embodiments, the diet comprises linoleic acid in an amount of about 8 g/Mcal. As used herein, the expression“x g/Mcal” for a given substance comprised in a diet means that the substance is comprised in an amount of x grams per Mcal contained in the diet. In certain embodiments, the diet comprises linoleic acid and zinc. In certain embodiments, the diet comprises zinc in an amount from about 40 mg/Mcal to about 60 mg/Mcal. In certain embodiments, the diet comprises zinc in an amount of about 50 mg/Mcal. Additional information on diets to improve skin conditions can be found in International Patent Publication No. WO 2020/055856, the content of which is incorporated by reference in its entirety.

Additional exemplary diets encompassed by the present disclosure can be found in International Patent Publication Nos. WO 2019/183557, WO 2019/144081, and U.S. Patent Publication No. US 2022/0096537, the content of each of which is incorporated by reference in its entirety.

### 4. EXAMPLES

The presently disclosed subject matter provides for improved accuracy of each subsystem in ancestry and trait classifications. Such classifications include but are not limited to local ancestry classification and global ancestry classification. The following describes the examples for these classifications.

### Example 1: Benchmarking Accuracy of Ancestry Classifiers

A publicly available dataset of 84,414 genetic variants genotyped in 4,368 dog samples from 87 breed groups was partitioned into a reference panel (n=4,168) and 200 single origin query samples. Furthermore, the 200 single origin query samples were then used to produce 200 highly admixed synthetic samples. Both the single origin and highly admixed query samples were subjected to local and global ancestry prediction in the presently disclosed system disclosed herein and RFMix. Since the true labels of the 200 query samples were known, the embodiments disclosed herein were able to compare the accuracy of the presently disclosed system with that of RFMix. The accuracy of the classifiers was measured as the mean squared error (MSE) between the predicted ancestry proportion and the true proportion.

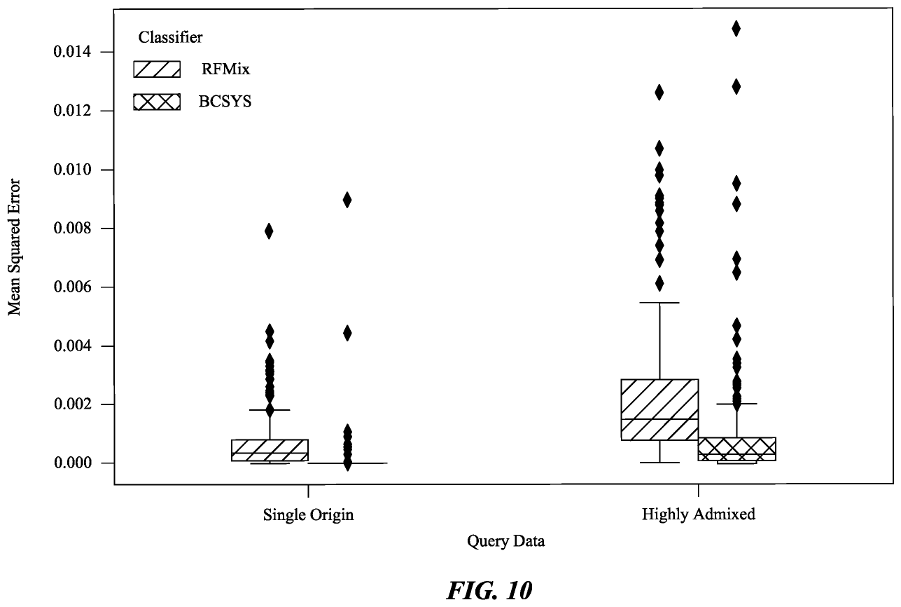

FIG. 10 illustrates example results of accuracy benchmark of our system versus the state-of-the-art classifier RFMix. FIG. 10 and Table 1 show the distribution of MSE for the 200 samples in each query set for RFMix and the presently disclosed system. For single origin query samples, both the presently disclosed system and RFMix showed similarly high levels of accuracy (FIG. 10). A paired sample t-test indicated no significant difference between MSE for our system compared to RFMix for single origin samples (t=−1.0749; P=0.2831). Conversely, the single origin and highly admixed sample average MSE were significantly different between the presently disclosed system and RFMix (t=14.1269; P<0.01).

TABLE 1
Average mean squared error for 200 samples using
the ancestry prediction system and RFMix.

| | | Average |
|---|---|---|
| System | Dataset | MSE |
| present disclosure | single origin | 0.000113 |
| RFMix | single origin | 0.000217 |
| present disclosure | highly admixed | 0.000009 |
| RFMix | highly admixed | 0.001647 |

### Example 2: Benchmarking Scalability of Classification System

The scalability of the presently disclosed system was compared to that of RFMix. Dramatic differences were observed in the compute resources utilized by the presently disclosed system and RFMix. To generate the results reported here, the presently disclosed system requires a maximum of 2 Gb of RAM and the entire workflow takes an average of 6 minutes to complete for all chromosomes. However, RFMix requires a maximum of 60 Gb of RAM and takes an average of 3 hours to complete a single chromosome data set. To run both workflows in a commodity cloud environment, RFMix would require a r5a.4xlarge instance type currently priced at $0.904 per hour and an average runtime of 3 hours to run a single chromosome data set for 200 samples. These requirements translate to a cost of $0.515 per sample. The requirements for the presently disclosed system are a m5.4xlarge instance type currently priced at $0.768 per hour, run for an average of 6 minutes for 200 samples, for all chromosomes means the price would be approximately $0.000384 per sample. It should be noted that RFMix was unable to accommodate reference panels with greater than 6,000 individuals, while the presently disclosed system was run efficiently with over 20,000 individual samples.

### Example 3: Assessing Accuracy of Global Ancestry Classification

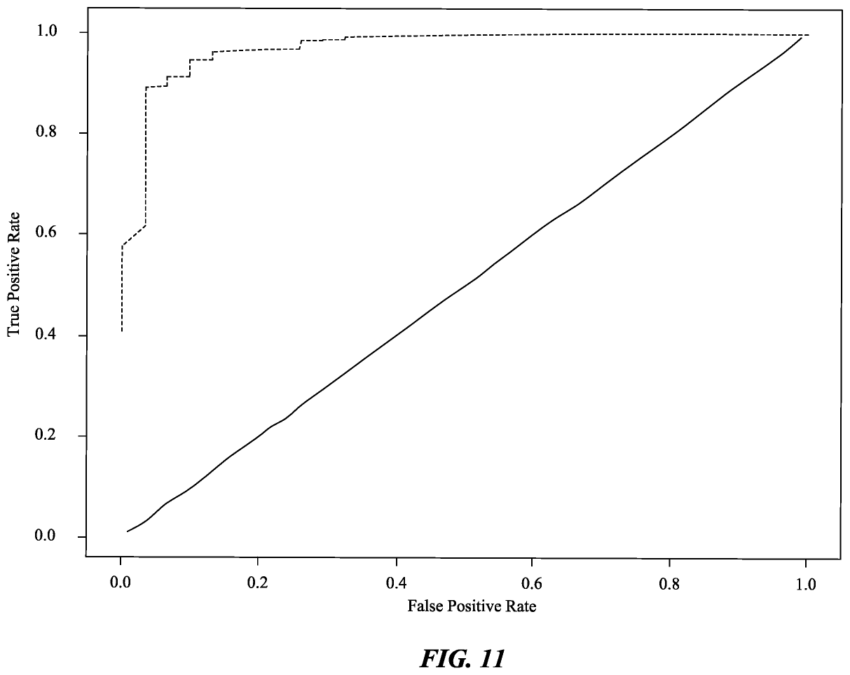

As described previously, conventional work cannot predict ancestry of a whole organism from local ancestry assignments. The embodiments disclosed herein have characterized the accuracy of the global ancestry classifier using a stratified k-fold cross-validation procedure. FIG. 11 illustrates an example receiver operating characteristic (ROC) curve for the global ancestry classifier. The macro recall using the publicly available reference panel is 0.9939 and the receiver operating characteristics (ROC) curve in FIG. 11 shows the area under curve (AUC) is 0.9192.

In addition to the organism label prediction, particular genetic variants can be used in conjunction with global ancestry to refine predictions. In a proof-of-concept experiment, 10 genetic markers known to have large phenotypic effects are used to further classify otherwise indistinguishable sub-types of Poodle (toy versus miniature), Collie (rough-haired versus smooth-haired), and Dachshund (long-haired versus short-haired). Table 2 shows the accuracy of using these additional markers in the context of a random forest machine learning model.

TABLE 2
Accuracy of predicting population sub-types based
on a limited set of genetic markers that are known
to have a large impact on animal phenotype.

| Comparison | Accuracy |
|---|---|
| Poodle types | 0.869 |
| Collie types | 0.951 |
| Dachshund types | 0.898 |

### Example 4: Performance of Trait Prediction

A decision tree-based machine learning approach has been employed in the predictor of traits suite to build a model capable of predicting healthy adult body weight in companion animals. The input to the machine learning algorithm is a 16,168 canine sample training set of global ancestry data, plus genotype data for 39 size and weight associated genetic markers, gender, neuter status and body weight data obtained from veterinary examination during hospital visits.

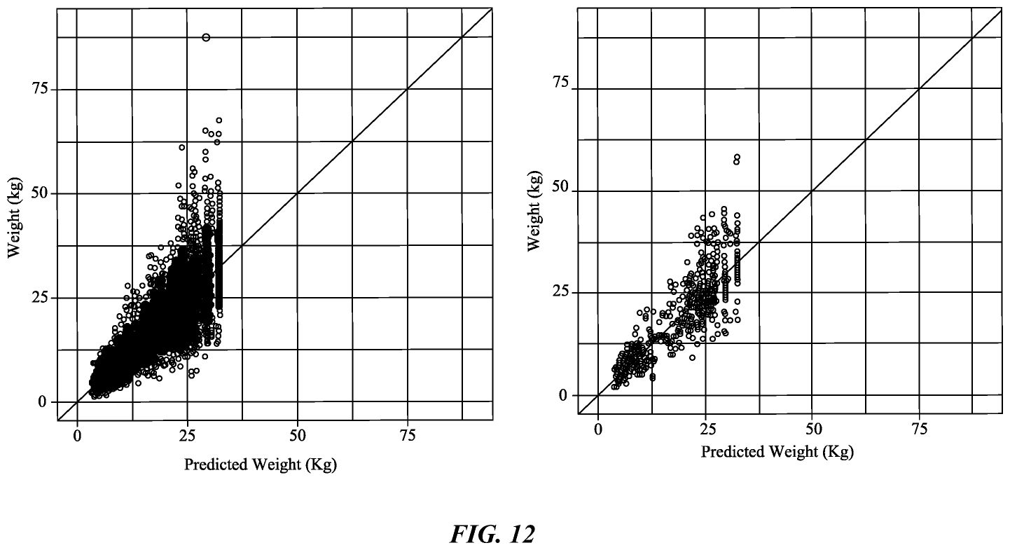

FIG. 12 illustrates an example regression of predicted adult body weight and true observed adult body weight. FIG. 12 shows an example regression analysis of predicted adult body weight and true-observed adult body weight using an example adult weight prediction module, according to the present embodiments and as discussed above. Evaluation of the body weight prediction model on a test set of samples yields a mean absolute percentage error (MAPE) of 21.8%.

### Example 5: Performance of Automated Accuracy Improvement

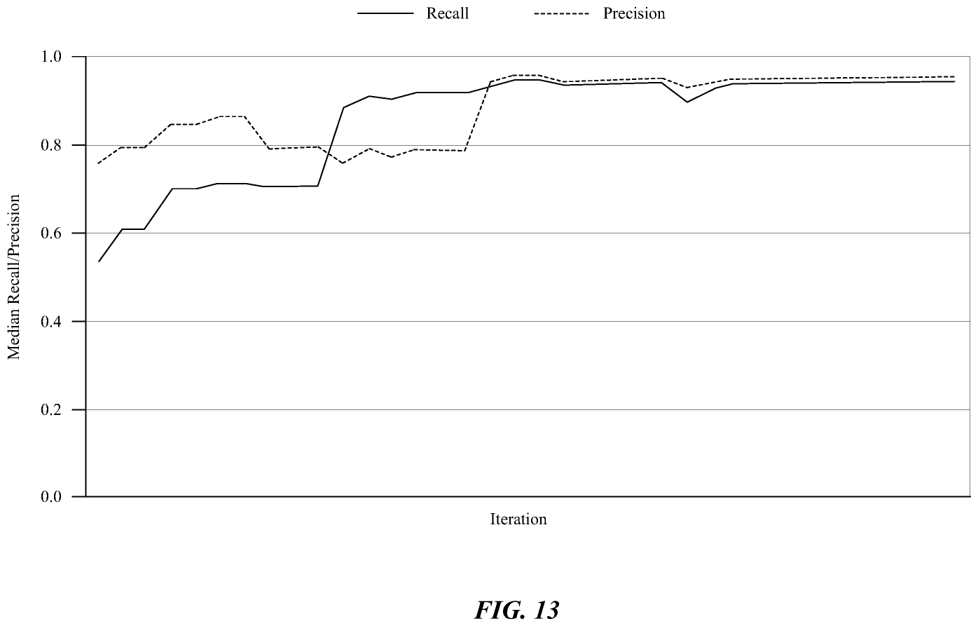

FIG. 13 illustrates an example iterative improvement of local ancestry reference panel using the isolation forest technique for anomaly detection. FIG. 13 shows that there is improvement in reference panel precision and recall upon application of further iterations of cross-validation methods, including isolation forest iteration, which remove erroneously labelled reference samples. In certain non-limiting embodiments, the cross-validation methods can be supervised or semi-supervised.

Table 3 illustrates the accuracy of distinguishing subtypes, as described above, using a supervised versus semi-supervised label propagation method, wherein the semi-supervised label propagation method was used to assign 50% of sub-type labels.

TABLE 3
Accuracy of distinguishing sub-types described
in the previous section, only with semi-supervised
label propagation to assign 50% of sub-type labels.

| Comparison | Supervised | Semi-Supervised |
|---|---|---|
| Poodle types | 0.869 | 0.869 |
| Collie types | 0.951 | 0.959 |

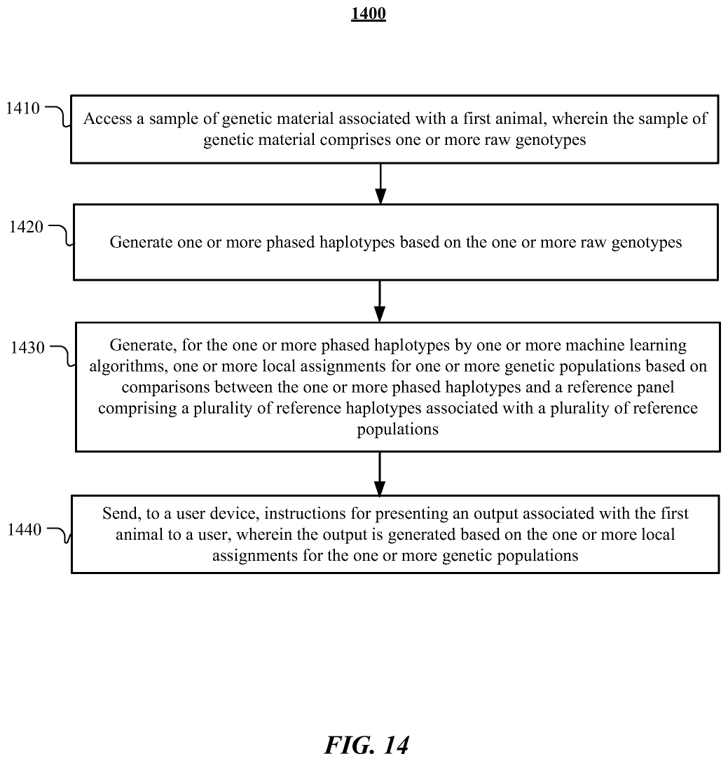

FIG. 14 illustrates an example method 1400 for ancestry prediction. The method can begin at step 1410, where the computing systems can access a sample of genetic material associated with a first animal, wherein the sample of genetic material comprises one or more raw genotypes. At step 1420, the computing systems can generate one or more phased haplotypes based on the one or more raw genotypes. At step 1430, the computing systems can generate, for the one or more phased haplotypes by one or more machine learning algorithms, one or more local assignments for one or more genetic populations based on comparisons between the one or more phased haplotypes and a reference panel comprising a plurality of reference haplotypes associated with a plurality of reference populations. At step 1440, the computing systems can send, to a user device, instructions for presenting an output associated with the first animal to a user, wherein the output is generated based on the one or more local assignments for the one or more genetic populations. Particular embodiments can repeat one or more steps of the method of FIG. 14, where appropriate. Although this disclosure describes and illustrates particular steps of the method of FIG. 14 as occurring in a particular order, this disclosure contemplates any suitable steps of the method of FIG. 14 occurring in any suitable order. Moreover, although this disclosure describes and illustrates an example method for ancestry prediction including the particular steps of the method of FIG. 14, this disclosure contemplates any suitable method for ancestry prediction including any suitable steps, which can include all, some, or none of the steps of the method of FIG. 14, where appropriate. Furthermore, although this disclosure describes and illustrates particular components, devices, or systems carrying out particular steps of the method of FIG. 14, this disclosure contemplates any suitable combination of any suitable components, devices, or systems carrying out any suitable steps of the method of FIG. 14.

Those skilled in the art will recognize that the methods and systems of the present disclosure can be implemented in many manners and as such are not to be limited by the foregoing exemplary embodiments and examples. In other words, functional elements being performed by single or multiple components, in various combinations of hardware and software or firmware, and individual functions, can be distributed among software applications at either the client level or server level or both. In this regard, any number of the features of the different embodiments described herein can be combined into single or multiple embodiments, and alternate embodiments having fewer than, or more than, all of the features described herein are possible.

Functionality can also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, as well as those variations and modifications that can be made to the hardware or software or firmware components described herein as would be understood by those skilled in the art now and hereafter.

Furthermore, the embodiments of methods presented and described as flowcharts in this disclosure are provided by way of example in order to provide a more complete understanding of the technology. The disclosed methods are not limited to the operations and logical flow presented herein. Alternative embodiments are contemplated in which the order of the various operations is altered and in which sub-operations described as being part of a larger operation are performed independently.

While various embodiments have been described for purposes of this disclosure, such embodiments should not be deemed to limit the teaching of this disclosure to those embodiments. Various changes and modifications can be made to the elements and operations described above to obtain a result that remains within the scope of the systems and processes described in this disclosure.

While the disclosed subject matter is described herein in terms of certain preferred embodiments, those skilled in the art will recognize that various modifications and improvements can be made to the disclosed subject matter without departing from the scope thereof. Moreover, although individual features of one non-limiting embodiment of the disclosed subject matter can be discussed herein or shown in the drawings of the one non-limiting embodiment and not in other embodiments, it should be apparent that individual features of one non-limiting embodiment can be combined with one or more features of another embodiment or features from a plurality of embodiments.

What is claimed is:

1. A method comprising, by one or more computing systems:
accessing a sample of genetic material associated with a first animal, wherein the sample of genetic material comprises one or more raw genotypes;
generating one or more phased haplotypes based on the one or more raw genotypes;
generating, for the one or more phased haplotypes by one or more machine learning algorithms, one or more local assignments for one or more genetic populations based on comparisons between the one or more phased haplotypes and a reference panel comprising a plurality of reference haplotypes associated with a plurality of reference populations;
determining, based on the one or more local assignments for the one or more genetic populations, one or more source populations associated with the first animal;
partitioning the one or more local assignments for the one or more genetic populations into one or more of a maternally-inherited group or a paternally-inherited group;
determining, based on the one or more local assignments for the one or more genetic populations and the one or more source populations, one or more genetic traits associated with the first animal; and
sending, to a user device, instructions for presenting an output associated with the first animal to a user, wherein the output is generated based on one or more of the one or more local assignments for the one or more genetic populations, the one or more source populations, results associated with the partitioning, or the one or more genetic traits.

2. The method of claim 1, wherein determining the one or more source populations comprises:
aggregating the one or more local assignments for the one or more genetic populations over both maternal and paternal chromosomes;
calculating proportions associated with the one or more source populations based on the aggregations; and
determining the one or more source populations based on the calculated proportions.

3. The method of claim 2, wherein the partitioning is based on one or more clustering algorithms.

4. The method of claim 1, wherein determining the one or more genetic traits is further based on one or more of genotypes of variants of large effect, genome-wide statistics, genomic principal component analysis (PCA) projections, DNA methylation profiles, or polygenic risk scores.

5. The method of claim 1, wherein the one or more genetic traits comprise one or more of:
a range of adult body weight;
a risk prediction or a predisposition to a genetic disease;
a nutrition recommendation;
a behavior and temperament class prediction;
a longevity estimation;
an all-causes mortality prediction in years;
a predicted pharmacological response; or
a recovery time range in hours for injectable anesthetics.

6. The method of claim 1, further comprising:
updating the one or more machine learning algorithms based one or more new reference samples added to the reference panel.

7. The method of claim 6, wherein the updating comprises:
applying a cross-validation across all samples in the reference panel;
identifying, based on results associated with the cross-validation by a detection algorithm, one or more outliers; and
removing the identified outliers from the reference panel.

8. The method of claim 6, wherein the updating is repeatedly iterated until a predetermined accuracy level of the one or more machine learning algorithms is reached.

9. The method of claim 6, wherein the updating further comprises:
generating one or more labels for one or more unlabeled samples in the reference panel, wherein the updating is based on the generated labels.

10. The method of claim 1, further comprising:
generating, based on the one or more raw genotypes, one or more consensus genotypes; and
generating, based on the one or more raw genotypes and the one or more consensus genotypes, the one or more phased haplotypes, wherein the generating comprises phasing the one or more raw genotypes and the one or more consensus genotypes into maternal and paternal chromosomes.

11. The method of claim 1, wherein the one or more machine learning algorithms comprise a positional Burrows-Wheeler transform algorithm.

12. The method of claim 1, further comprising:
removing one or more errors associated with the one or more local assignments for the one or more genetic populations based on the one or more machine learning algorithms.

13. The method of claim 1, wherein the one or more machine learning algorithms comprise a hidden Markov model.

14. One or more computer-readable non-transitory storage media embodying software that is operable when executed to:
access a sample of genetic material associated with a first animal, wherein the sample of genetic material comprises one or more raw genotypes;
generate one or more phased haplotypes based on the one or more raw genotypes;
generate, for the one or more phased haplotypes by one or more machine learning algorithms, one or more local assignments for one or more genetic populations based on comparisons between the one or more phased haplotypes and a reference panel comprising a plurality of reference haplotypes associated with a plurality of reference populations;
determine, based on the one or more local assignments for the one or more genetic populations, one or more source populations associated with the first animal;
partition the one or more local assignments for the one or more genetic populations into one or more of a maternally-inherited group or a paternally-inherited group;
determine, based on the one or more local assignments for the one or more genetic populations and the one or more source populations, one or more genetic traits associated with the first animal; and
send, to a user device, instructions for presenting an output associated with the first animal to a user, wherein the output is generated based on one or more of the one or more local assignments for the one or more genetic populations, the one or more source populations, results associated with the partitioning, or the one or more genetic traits.

15. The media of claim 14, wherein determining the one or more source populations comprises:
aggregating the one or more local assignments for the one or more genetic populations over both maternal and paternal chromosomes;
calculating proportions associated with the one or more source populations based on the aggregations; and
determining the one or more source populations based on the calculated proportions.

16. The media of claim 15, wherein the partitioning is based on one or more clustering algorithms.

17. The media of claim 14, wherein determining the one or more genetic traits is further based on one or more of genotypes of variants of large effect, genome-wide statistics, genomic principal component analysis (PCA) projections, DNA methylation profiles, or polygenic risk scores.

18. The media of claim 14, wherein the one or more genetic traits comprise one or more of:
a range of adult body weight;
a risk prediction or a predisposition to a genetic disease;
a nutrition recommendation;
a behavior and temperament class prediction;
a longevity estimation;
an all-causes mortality prediction in years;
a predicted pharmacological response; or
a recovery time range in hours for injectable anesthetics.

19. The media of claim 14, wherein the software is further operable when executed to:
update the one or more machine learning algorithms based one or more new reference samples added to the reference panel.

20. The media of claim 19, wherein the updating comprises:
applying a cross-validation across all samples in the reference panel;
identifying, based on results associated with the cross-validation by a detection algorithm, one or more outliers; and
removing the identified outliers from the reference panel.

21. The media of claim 19, wherein the updating is repeatedly iterated until a predetermined accuracy level of the one or more machine learning algorithms is reached.

22. The media of claim 19, wherein the updating further comprises:
generating one or more labels for one or more unlabeled samples in the reference panel, wherein the updating is based on the generated labels.

23. The media of claim 14, wherein the software is further operable when executed to:
generate, based on the one or more raw genotypes, one or more consensus genotypes; and
generate, based on the one or more raw genotypes and the one or more consensus genotypes, the one or more phased haplotypes, wherein the generating comprises phasing the one or more raw genotypes and the one or more consensus genotypes into maternal and paternal chromosomes.

24. The media of claim 14, wherein the one or more machine learning algorithms comprise a positional Burrows-Wheeler transform algorithm.

25. The media of claim 14, wherein the software is further operable when executed to:
remove one or more errors associated with the one or more local assignments for the one or more genetic populations based on the one or more machine learning algorithms.

26. The media of claim 14, wherein the one or more machine learning algorithms comprise a hidden Markov model.

27. A system comprising: one or more processors; and a non-transitory memory coupled to the processors comprising instructions executable by the processors, the processors operable when executing the instructions to:
access a sample of genetic material associated with a first animal, wherein the sample of genetic material comprises one or more raw genotypes;
generate one or more phased haplotypes based on the one or more raw genotypes;
generate, for the one or more phased haplotypes by one or more machine learning algorithms, one or more local assignments for one or more genetic populations based on comparisons between the one or more phased haplotypes and a reference panel comprising a plurality of reference haplotypes associated with a plurality of reference populations;
determine, based on the one or more local assignments for the one or more genetic populations, one or more source populations associated with the first animal;
partition the one or more local assignments for the one or more genetic populations into one or more of a maternally-inherited group or a paternally-inherited group;
determine, based on the one or more local assignments for the one or more genetic populations and the one or more source populations, one or more genetic traits associated with the first animal; and
send, to a user device, instructions for presenting an output associated with the first animal to a user, wherein the output is generated based on one or more of the one or more local assignments for the one or more genetic populations, the one or more source populations, results associated with the partitioning, or the one or more genetic traits.

28. The system of claim 27, wherein the processors are further operable when executing the instructions to:
update the one or more machine learning algorithms based one or more new reference samples added to the reference panel.

29. The system of claim 28, wherein the updating comprises:
applying a cross-validation across all samples in the reference panel;
identifying, based on results associated with the cross-validation by a detection algorithm, one or more outliers; and
removing the identified outliers from the reference panel.

30. The system of claim 27, wherein the processors are further operable when executing the instructions to:
remove one or more errors associated with the one or more local assignments for the one or more genetic populations based on the one or more machine learning algorithms.
